(19) United States
(12) Patent Application Publication
(10) Pub. No.: US 2023/0193213 A1
(43) Pub. Date: Jun. 22, 2023

(54) RECOMBINANT ONCOLYTIC NEWCASTLE DISEASE VIRUSES WITH INCREASED ACTIVITY

(71) Applicant: Arno THALLER, Markt Berolzheim (DE)

(72) Inventor: Arno THALLER, Markt Berolzheim (DE)

(21) Appl. No.: 17/419,792

(22) PCT Filed: Oct. 7, 2019
(86) PCT No.: PCT/EP2019/077104

§ 371 Date: Jun. 30, 2021

(30) Foreign Application Priority Data
Jan. 29, 2019 (EP) 19154204.2

(51) Int. Cl. C12N 7/00; C12N 15/86; C07K 14/005

(52) U.S. Cl. CPC C12N 7/00; C12N 15/86; C07K 14/005; C12N 2760/18121; C12N 2760/18122; C12N 2760/18132

(57) ABSTRACT

The invention relates to transgene expressing Newcastle Disease Viruses (NDV), which have been demonstrated to possess significant oncolytic activity against mammalian cancers. The invention provides novel oncolytic viruses through the use of genetic engineering, including the transfer of foreign genes or parts thereof, such as genes encoding Ipilimumab, interleukin-12 or NS1. The present invention also provides nucleic acids encoding a reverse genetically engineered (rg-)NDV comprising one or more of these foreign genes and having a mutation in the HN gene, said mutation allowing replication of said rgNDV in a cancer cell to a higher level than replication of an otherwise identical rgNDV not having said mutation in the HN gene.

This invention relates to recombinant Newcastle Disease Viruses (NDV), which have been demonstrated to possess significant oncolytic activity against mammalian cancers, especially selected from the specific cancers as described herein and/or mentioned in the claims. The invention provides the development of novel and improved oncolytic agents through the use of genetic engineering, including the transfer of genes across species boundaries in order to produce improved genetically modified viruses carrying these transgenes.

### BACKGROUND

NDV is known as an oncolytic virus that is a virus for use in oncological treatment, preferably in the treatment of human subjects in need thereof. A number of RNA viruses, including NDV, reovirus, measles virus, and vesicular stomatitis virus (VSV), are members of this novel class of viruses being exploited as potential oncolytic agents. These oncolytic viruses are characterized in inherently replicating selectively within and killing tumor cells while leaving non-tumor cells unharmed. Accordingly these oncolytic viruses offer an attractive new tool for cancer therapy.

NDV derives its name from the site of the original outbreak in chickens at a farm near Newcastle-upon-Tyne in England in 1926. The virus is an economically important pathogen in multiple avian species and it is endemic in many countries. NDV is a member of the Avulavirus genus in the Paramyxoviridae family and is a non-segmented, negative-strand RNA virus, whose natural host range is limited to avian species; however, it is known to enter cells by binding to sialic acid residues present on a wide range of human and rodent cancer cells. The oncolytic property of NDV, i.e. to selectively replicate in and destroy tumor cells, while sparing normal cells, is believed to be based in part on defective antiviral responses in tumor cells. Normal cells, which are competent in launching an efficient antiviral response rapidly after infection, are able to inhibit viral replication before viral-mediated cell damage can be initiated. The sensitivity of NDV to interferon (IFN), coupled with defective IFN signalling pathways in tumor cells, provides one plausible mechanism whereby NDV can replicate exclusively within neoplastic tissue. The selective replication of NDV in human tumor cells may also be caused by several other mechanisms, including defects in activation of anti-viral signalling pathways, and activation of Ras signalling and/or expression of Racl (Schirrmacher, 2015, Expert Opin. Biol. Ther. 15:17 57-71).

Due to its tumor-specifity NDV is already under investigation as a safe clinical oncolytic virotherapy agent (Cassel et al., 1965, Cancer 1:863-888; Steiner et al., 2004, J. Clin. Oncol. 22:4272-4281; Schulze et al., 2009, Cancer Immunol. Immunother. 58:61-69; Csatary et al., 1999, Anticancer Res. 19:635-638; Pecora et al., 2002, J. Clin. Oncol. 20:2251-2266; Lorence et al., 2003, Curr. Opin. Mol. Ther. 5:618-624; Freeman et al., 2006, Mol. Ther. 13:221-228). The safety margin of NDV is a function of its inability to replicate and kill normal human cells.

Typically, an oncolytic NDV strain is defined as an NDV for use in oncological virotherapy, preferably in the treatment of human subjects in need thereof. Multiple preclinical model studies have shown significant anti-tumor activity of natural and recombinant oncolytic NDV strains after varying treatment modalities. Promising results were noted in preclinical models of glioblastoma multiforme, anaplastic astrocytoma, leukemia, lymphoma, melanoma, neuroblastoma, osteosarcoma, rhabdomyosarcoma, Ewing's sarcoma, fibrosarcoma, pheochromocytoma, colon carcinoma, lung carcinoma, prostate carcinoma, breast carcinoma, ovary carcinoma, gastric carcinoma, mesothelioma, renal cell carcinoma, and head and neck carcinoma. One typically useful oncolytic NDV that has been extensively explored for use in the treatment of humans, and for which an advantageous safety and efficacy profile has been established in human trials, is a strain named MTH-68/H (Csatary L K, et al., J Neurooncol. 2004 March-April; 67(1-2):83-93). MTH-68/H therapy has been employed in a range of different tumors with success. MTH-68/H has been developed into a highly purified, lyophilized product, containing live, replication competent viral particles, grown to standardized titers. A Phase II clinical trial in humans was completed where the inhalatory mode of administration was used on patients suffering from a variety of advanced malignancies no longer responding to conventional cancer therapies. The study demonstrated relative efficacy, an overall improved quality of life, and a relatively benign side effect profile (Csatary L K, et al., Cancer detection and Prevention, 1993, 17(6):619-627). Success of treatment with MTH-68/H has also been shown in glioblastomas and anaplastic astrocytomas. Glioblastoma multiforme (GBM) is the most common primary brain tumor and by far the most aggressive form of glial tumors. This neoplasm has a poor prognosis, averaging six months to a year without therapy or about one-and-one-half years with conventional therapy. Four cases of advanced GBM and anaplastic astrocytoma were treated with the NDV viral product MTH-68/H after the conventional modalities of cancer therapy had failed. Results included survival rates of twelve and half to six years post-viral treatment for the surviving patients, and six years for one patient who has since died, after having discontinued treatment. Against all odds, these surviving patients returned to good quality of life and to a lifestyle that resemble their pre-morbid daily routines including giving birth to a healthy child and full professional activity. They have been able to enjoy good clinical health. These patients regularly received the MTH-68/H viral therapy for a number of years without interruption as a form of monotherapy once the classical modalities failed. The MRI and CT results have revealed an objective decrease in size of the tumors, in some cases the near total disappearance of the tumor mass.

As reviewed by Zamarin and Palese (Future Microbiol. 2012 March; 7(3): 347-367), when compared to other oncolytic agents in development such as poxvirus, HSV-1, adenovirus, measles, and reovirus vectors, NDV as an oncolytic virus exhibits several advantages over other viruses for use in oncological treatment.

The first advantage is that NDV is an avian pathogen. This avoids the problems of preexisting immunity that can neutralize virus infectivity and pathogenicity of the virus in humans. These potential problems exist when using vaccinia, HSV-1, adenovirus, and measles vectors. Based on serological studies it seems that about 96% of the human population is seronegative for NDV, which avoids the problem of pre-existing immunity. The actual level of seropositivity in today's population may be even lower due to low human exposure to NDV, as the outbreaks of which are primarily limited to farm settings. To attest for viral safety, administration of virulent strains to humans has been shown to result in only mild to moderate adverse effects, with mild conjunctivitis, laryngitis, and flu-like symptoms as the only reported side effects. Natural human infections with highly virulent (avian) strains have been reported in the literature in farmers and laboratory workers and have been limited to mild symptoms such as conjunctivitis and laryngitis.

The second advantage offered by using NDV as an oncolytic agent is that NDV, similarly to other oncolytic viruses, possesses strong immunostimulatory properties that are the basis for oncolytic therapy being considered an immunotherapy. These properties include the induction of type I IFN and chemokines, upregulation of MHC and cell adhesion molecules, and facilitation of adhesion of lymphocytes and antigen-presenting cells (APCs) through expression of viral glycoproteins on the surface of infected cells. These properties have been shown to generate effective anti-tumor immune responses, which may persist long after clearance of the primary viral infection.

A third advantage is that the NDV genome has the plasticity to enable the incorporation and stable expression of foreign genes of relatively large size. Since these viruses replicate in the cytoplasm of the cell and not in the nucleus, an unintended integration of the foreign gene into the host genome is avoided. Such an unintended integration of foreign genes carried by an oncolytic agent into the host genome could be a safety problem with some DNA oncolytic vectors. Moreover, the absence of homologous RNA recombination ensures that foreign genes incorporated in and expressed from the NDV genome are stable for many serial passages in cell culture and in tumor cells.

Lastly, the ubiquitous nature of the NDV receptor allows for utilization of the virus against a wide variety of tumor types. The specificity of NDV for cancer cells due to their defects in antiviral pathways ensures viral safety.

Nevertheless, the full view of all clinical data generated to date in different trials of different NDV strains shows that there is much need for improvement in terms of percentage responders and magnitude of therapeutic effect. This need for improvement has spurred efforts to design improved recombinant NDV strains, which is the subject of the present patent application.

Similar to other paramyxoviruses in its family, the 15186, 15192 or 15198 nucleotide(nt)-long negative single-strand RNA genome of NDV encodes six genes including the nucleocapsid protein (NP), phosphoprotein (P), matrix protein (M), fusion protein (F), hemagglutinin-neuraminidase (HN), and RNA-dependent RNA polymerase (L) (Lamb R, Paramyxoviridae: the viruses and their replication. In: Knipe D, editor. Fields Virology, Lippincott Williams & Wilkins; 2007, pp. 1449-1496). The genes are separated by junction sequences that consist of three elements, known as gene start (GS), intergenic (IG), and gene-end (GE) motifs, which regulate mRNA transcription. In the P gene, a unique RNA editing mechanism adds non-templated G residues, resulting in the expression of V and W proteins that are colinear to the P protein in the amino-terminal end. The genomic RNA is bound in a ribonucleotide protein complex (RNP) consisting of NP, P, and L proteins and is surrounded by a lipid envelope containing three virus glycoprotein spikes, HN, M and F. An important factor that influences the extent of virulence of NDV in birds is the cleavage site of the F protein.

Pathogenic classification and virulence of NDV strains in birds generally correlates with their oncolytic properties in human cancer cells. Velogenic (high virulence) strains produce severe respiratory and nervous system signs, spread rapidly through chicken flocks, and can cause up to 90% mortality. Mesogenic (intermediate virulence) strains cause coughing, affect egg production and quality, and can result in up to 10% mortality. Lentogenic (low virulence) strains produce only mild symptoms with little if any mortality. Correspondingly, velogenic strains can efficiently carry out multicycle replication in multiple human cancer cells with effective and efficient cell lysis, lentogenic strains are more attenuated due to lack of activation of the FO protein, and mesogenic strains convey intermediate effects. On the basis of these observations in human cancers, NDV strains have been classified as either lytic or non-lytic, with velogenic and mesogenic viruses being lytic (high and low respectively), and lentogenic viruses in general being non-lytic. Early studies demonstrated that the lytic abilities of lentogenic NDV strains could be enhanced by the introduction of a polybasic cleavage site into their F proteins (Peeters et al, J. Virol. 1999; 73:5001-5009). Other NDV proteins including NP, P, V, HN, and L have also been shown to be implicated in virulence in birds. A deletion (18nt) introduced in the NP gene (Mebatsion T et al., J Virol. 2002 October; 76 (20):10138-46) resulted in the deletion of NP residues 443-460. The resulting mutant NDV propagated in embryonated specific-pathogen-free (SPF) chicken eggs to a level fully comparable to that of the parent virus.

In addition, a B-cell epitope of the S2 glycoprotein of murine hepatitis virus (MHV) was inserted in-frame. Recombinant NDV viruses properly expressing the introduced MHV epitope were successfully generated, demonstrating that the NP can be used as the insertion point in the NDV genome to insert foreign or transgenic sequences to be co-expressed with NDV during virus infection.

Several studies showed that type I IFN antagonist activity of NDV V protein is associated with pathogenicity in birds. In recombinant viruses possessing attenuating mutations or deletions in the V protein or in viruses expressing no or lower levels of V protein decreased pathogenicity in birds was observed (Huang et al., 2003, J Virol. 77:8676-8685; Mebatsion et al., 2001, J Virol. 75:420-428; Qiu et al., 2016, PLoS ONE 11(2): e0148560. doi: 10.1371/journal.pone.0148560; Elankumaran et al., 2010, J Virol. 84:3835-3844; Jang et al., 2010, Appl Microbiol Biotechnol. 85:1509-1520; Alamares et al., 2010, Virus Res. 147:153-157; Park et al., 2003, J Virol. 77:9522-9532.). It was found to be sufficient to introduce a small (6 nt) deletion in the P gene to abolish expression of the V protein (Mebatsion et al., 2001).

Recently, two studies showed the role of the polymerase complex in viral pathogenesis, with L protein contributing to pathogenicity of the mesogenic Beaudette C strain, and NP, P, and L proteins contributing to pathogenicity of the velogenic Herts strain (Rout & Samal, 2008, J Virol. 82:7828-7836; Dortmans et al., 2010, J Virol. 84:10113-10120). In addition, several studies noted the contribution of the HN protein to virulence in birds (Römer-Oberdörfer et al., 2006, Avian Dis. 50:259-263; Paldurai et al., 2014, J Virol. 88: 8579-8596.) The HN (hemagglutinin-neuraminidase) protein of NDV is a multifunctional protein with receptor-recognition, hemagglutinin (HA) and receptor-destroying neuraminidase (NA) activities associated with the virus. HN is thought to possess both the receptor recognition of sialic acid at the termini of host glycoconjugates and the neuraminidase activity to hydrolyze sialic acid from progeny virion particles in order to prevent viral self-aggregation. It also recognizes sialic acid-containing receptors on cell surfaces and promotes the fusion activity of the F protein, thereby allowing the virus to penetrate the cell surface. Thus, the HN protein plays a critical role in viral infection of birds.

A recombinant NDV vector virus can be provided by a reverse genetics (rg) system, which has been known since 1999 (Peeters et al., 1999, J. Virol. 73:5001-5009). This system allows desired modification and engineering of NDV genomes. Indeed, various reports have shown the successful use of recombinant NDV vectors engineered to express various transgenes, i.e. foreign genes incorporated in the genome of the virus, with the goal of improving viral oncolytic efficacy (Vigil et al. Cancer Res. 2007, 67:8285-8292; Janke et al., 2007, Gene Ther. 14:1639-1649). However, it should be noted that the replication capability of a recombinant NDV expressing a transgene may be hampered in comparison with its parental NDV strain not expressing the transgene. As discussed above, the viral F protein of NDV is responsible for viral fusion with the cell membrane and for viral spread from host cell to host cell via formation of syncytia. The presence of the multi-basic amino acid cleavage site in the F protein enables protein cleavage and activation by a broad range or proteases and is known to be a determinant of virulence in velogenic NDV strains. In order to increase the oncolytic potency of a highly attenuated lentogenic Hitchner B1 NDV strain, a polybasic cleavage site was introduced into the F protein to generate rNDV/F3aa with mutated F protein. Altomonte et al. (Mol Ther. 2010 18:275-84) reported an oncolytic NDV vector virus with the mutated F-protein rNDV/F(L289A), harboring an L289A mutation within the F protein, which is required for virus entry and cell-cell fusion. This rNDV/F3aa(L289A) virus with mutated F protein showed further enhanced fusion and cytotoxicity of hepatocellular carcinoma (HCC) cells in vitro, as compared with a rNDV/F3aa control virus. In vivo administration of rNDV/F3aa(L289A), via hepatic arterial infusion in immune-competent Buffalo rats bearing multifocal, orthotopic liver tumors resulted in tumor-specific syncytia formation and necrosis, with no evidence of toxicity to the neighboring hepatic parenchyma, which translated to a 20% prolongation of survival time relative to treatment with the original rNDV/F3aa virus.

Due to compelling preclinical data implicating oncolytic NDV as an ideal candidate for cancer therapy, phase I and II clinical trials have been initiated. Several trials have shown that NDV is a safe and potentially therapeutically useful therapeutic agent, with no reports of significant adverse effects in patients beyond conjunctivitis or mild flu-like symptoms (Csatary et al., 1999, Anticancer Res. 19:635-638; Pecora et al., 2002, J. Clin. Oncol. 20:2251-2266; Lorence et al., 2003, Curr. Opin. Mol. Ther. 5, 618-624; Freeman et al., 2006, Mol. Ther. 13:221-228). Although the results of these clinical trials have been encouraging, the extent of clinical responses has not been strong and robust enough to consider bringing one of these initial NDV strains into advanced clinical development. Thus, strategies for improving therapeutic effectiveness while maintaining an acceptable safety profile of oncolytic NDV strains are needed. Thus there remains a clear need for improvement in therapeutic outcome by providing improved NDV strains, in particular NDV strains with enhanced killing effect without affecting its safety toward normal cells.

### DESCRIPTION OF THE PRESENT INVENTION

In a first aspect the present invention provides recombinant Newcastle disease virus or NDV strains. A recombinant NDV according to the present invention carries as a foreign gene at least one gene selected from the group consisting of:
A gene encoding an antibody directed to the surface protein CTLA-4 or an antigen-binding part directed to the surface protein CTLA-4 (anti-CTLA-4), such as Ipilimumab or an antigen-binding part of Ipilimumab, or a functional analogs thereof.
A gene encoding a protein which improves the cellular immune response and improves the ability of T cells to enter tumor cells, or a part thereof which improves the cellular immune response and improves the ability of T cells to enter tumor cells, such as interleukin-12 (IL-12) or a part of interleukin-12, or functional analogs thereof.
A gene encoding a protein with the ability to modulate the virus replication cycle, or a part thereof with the ability to modulate the virus replication cycle, such as the non-structural protein NS1 of influenza A virus or a part or functional analogs thereof.
Any combination of these genes, parts or functional analogs of these genes or parts thereof.

The foreign gene or the parts or functional analogs thereof is/are expressed in the recombinant NDV strain of the present invention, when the virus is replicating in a suitable host or host cell. An expression of a gene means that the nucleic acid information encoded by the gene is translated in a respective amino acid sequence, which is the primary sequence for building up the respective protein or gene product. By way of example, the gene product of the HN gene is the HN protein.

Within the present invention the foreign gene carried by the recombinant NDV encodes in one embodiment an antibody capable of blocking checkpoint inhibition. The antibody is directed to the surface protein CTLA-4 (cytotoxic T-lymphocyte-associated protein 4), blocking the inhibitory signal, which allows T cells to have a higher activity towards tumor cells. In an alternative the foreign gene carried by the recombinant NDV encodes an antigen-binding part directed to protein CTLA-4. According to the present invention the antibody or antigen-binding part thereof is in a particularly preferred embodiment Ipilimumab, an antigen-binding part of Ipilimumab or a functional analog of Ipilimumab or a functional analog of an antigen-binding part of Ipilimumab.

According to another embodiment of the present invention the foreign gene carried by the recombinant NDV encodes a gene encoding a protein which improves the cellular immune response and improves the ability of T cells to enter cancer cells, or a part thereof which improves the cellular immune response and improves the ability of T cells to enter tumor cells. According to a preferred embodiment of the present invention the protein is interleukin-12 (IL-12) or a part of interleukin-12 or a functional analog of interleukin-12 or a functional analog of a part of interleukin-12.

According to another embodiment of the present invention the foreign gene carried by the recombinant NDV encodes a gene encoding a protein with the ability to modulate the virus replication cycle, or a part thereof with the ability to modulate the virus replication cycle. In a preferred embodiment the encoded protein is the non-structural protein NS1 of influenza A virus or a part of the non-structural protein NS1 of influenza A virus or a functional analog of the non-structural protein NS1 of influenza A virus or a functional analog of a part of the non-structural protein NS1 of influenza A virus.

In another embodiment of the present invention a recombinant NDV comprises in its viral genome at least two, preferably three, foreign genes or parts thereof, which foreign genes are selected from a gene encoding an antibody or its antigen-binding part directed to the surface protein CTLA-4 or functional analogs thereof (anti-CTLA-4), preferably Ipilimumab or an antigen-binding part or functional analogs thereof, a gene encoding a protein which improves the cellular immune response and improves the ability of T cells to enter cancer cells or a part thereof, preferably interleukin-12 (IL-12) or a part of interleukin-12 or functional analogs thereof, and a gene encoding a protein with the ability to modulate the virus replication cycle or a part thereof, preferably the non-structural protein NS1 of influenza A virus or a part of the non-structural protein NS1 of influenza A virus or functional analogs thereof.

As used herein a “functional analog” is a variant of a starting sequence. A variant of a starting nucleic acid is a nucleic acid that comprises a nucleic acid sequence different from that of the starting nucleic acid. Preferably, a variant will possess at least 75% sequence identity, more preferably at least 90% sequence identity, still more preferably at least 95% sequence identity, and most preferably at least 98% sequence identity with the native nucleic acid. Variants of a nucleic acid may be prepared by introducing appropriate nucleotide changes into the nucleic acid. Such variants include, for example, deletions from, and/or insertions into and/or substitutions of residues within the nucleic acid sequence of the gene of interest. Any combination of deletion, insertion, and substitution is possibly made to arrive at the final construct, provided that the final construct possesses the desired characteristics. Methods for generating nucleic acid sequence variants are known to the skilled person.

A variant of a starting polypeptide or protein, e.g. an antibody or another protein, is a polypeptide or protein that comprises an amino acid sequence different from that of the starting polypeptide or protein. Preferably, a variant will possess at least 75% sequence identity, more preferably at least 90% sequence identity, still more preferably at least 95% sequence identity, and most preferably at least 98% sequence identity with the native polypeptide or protein. Variants of a polypeptide or a proteine may be prepared by introducing appropriate nucleotide changes into the nucleic acid encoding the polypeptide or protein. Such variants include, for example, deletions from, and/or insertions into and/or substitutions of residues within the amino acid sequence of the polypeptide/proteine of interest. Any combination of deletion, insertion, and substitution is possibly made to arrive at the final construct, provided that the final construct possesses the desired characteristics. The amino acid changes also may alter post-translational processes of the polypeptide, such as changing the number or position of glycosylation sites. Methods for generating amino acid sequence variants of polypeptides are known to the skilled person.

As used herein, percentage sequence identity is preferably determined after the best alignment of the sequence of interest with the respective reference sequence. The best alignment of the sequences may for example be produced by means of the Fitchet al., Proc. Natl. Acad. Sci. USA 80:1382-1386 (1983), version of the algorithm described by Needleman et al., J. Mol. Biol. 48:443-453 (1970), after aligning the sequences to provide for best homology, by means of the similarity search method of Pearson and Lipman, Proc. Natl Acad. Sci. USA 85, 2444 (1988), or by means of computer programs which use these algorithms (in particular BLASTN in Wisconsin Genetics Software Package, Genetics Computer Group, 575 Science Drive, Madison, Wis.). The percentage identity is calculated by determining the number of identical positions between the two sequences being compared, dividing this number by the number of positions compared and multiplying the result obtained by 100 so as to obtain the percentage identity between these two sequences. Preferably, sequence identity of a nucleotide sequence is determined using BLASTN, preferably BLASTN in standard settings as provided by the website of the U.S. National Library of Medicine “https://blast.ncbi.nlm.nih.gov”. Also preferably, sequence identity of an amino acid sequence is determined using BLASTP, preferably BLASTP in standard settings as provided by the website of the U.S. National Library of Medicine “https://blast.ncbi.nlm.nih.gov”. It is further preferred that the sequence identity is calculated over the entire length of the respective sequence.

As used herein, the term “part” means a fragment of a starting nucleic acid or amino acid sequence, which fragment is a made up of a consecutive sequence of the nucleotides or amino acids of the starting sequence. The part or fragment preferably comprises at least 10, more preferably at least 20 and preferably up to 500 nucleotides or amino acids. In any case a “part” according to the present invention is a functional part. That is nucleic acid or proteine encoded by that nucleic acid must provide the desired function, e.g. the antigen-binding function or improving the cellular immune response and the ability of T cells to enter tumor cells or modulating the virus replication cycle.

In addition to carrying at least one foreign gene or a part or functional analog thereof as specified supra, the recombinant NDV strains according to the present invention further comprise a mutation in the viral HN gene leading to a change in the amino acid sequence of the HN protein and providing the NDV with an increased replication capability in a cancer cell, preferably in a human cancer cell, as compared to an otherwise identical NDV not having the said mutation in the HN gene.

Preferably the mutation in the HN gene results in an unexpected beneficially increased replication capability of the recombinant NDV strain in cancer cells, preferably in human cancer cells, and more preferably results in a replication of said oncolytic NDV in a human cancer cell which is at least 2-fold higher, more preferably 2- to 10-fold higher, still more preferably 5- to 10-fold higher than an NDV parent strain not having said mutation in its viral HN gene. Preferably, said parent strain comprises or is an oncolytic NDV already having an advantageous safety and efficacy profile in humans, such as the NDV strain MTH-68/H (SEQ ID No. 1) or a functional analog of the NDV strain MTH-68/H.

According to a preferred embodiment the recombinant NDV strain comprises a mutated HN gene and the encoded hemagglutinin-neuramidase (HN protein) with an amino acid substitution at position 277 to an amino acid with a hydrophobic side chain other than phenylalanine. Still more preferred is the amino acid phenylalanine (F) substituted to leucine (L) at amino acid position 277 of the HN gene. In a more preferred embodiment, the invention provides an oncolytic NDV derived from NDV strain MTH-68/H having this mutation in the HN gene and carrying at least one of the foreign genes or parts or functional analogs thereof as specified supra, said oncolytic NDV according to the invention having an unexpected beneficially increased replication capability allowing replication of said oncolytic NDV in a human cancer cell preferably up to 10-fold higher levels than the oncolytic NDV parent strain MTH-68/H, thereby maintaining the useful oncological safety and efficacy specifications of the parent virus and advantageously supplementing it with the increased replication capability of an oncolytic NDV and the ability to express within infected cancer cells at least one of the before mentioned foreign genes, which beneficialls influence the immunological response towards the cancer cells. Herein an oncolytic NDV having such a mutation is also identified as NDV^{F277L} to discern it from a parent NDV having a phenylalanine at position 277 of the HN gene.

A NDV strain derived from NDV strain MTH-68/H having a mutation in the HN gene at amino acid position 277, wherein the amino acid phenylalanine (F) substituted to leucine (L) at position 277 is referred to herein also as NDV-Mut HN(F277L). The foreign gene or part or respective functional analog thereof carried by this strain is referred to as Ipilimumab, IL-12 or NS1 in this shortcut for the strain. Within the shortcut for the recombinant NDV strains as used herein a reference to Ipilimumab is intended to comprise the complete antibody, antigen-bindings parts and functional analogs of the antibody or functional analogs of an antigen-binding part thereof, but also comprises the more generic form as described herein, i.e. an antibody directed to the surface protein CTLA-4 or an antigen-binding part of this antibody. The same applies for the other foreign genes. That is a recombinant NDV strain referred to as “NDV-Mut HN(F277L)-Ipilimumab” is a NDV strain derived from NDV strain MTH-68/H having a mutation in the HN gene at amino acid position 277, wherein the amino acid phenylalanine (F) is substituted to leucine (L) at position 277, and carrying as a foreign gene a gene encoding an antibody that is directed to the surface protein CTLA-4 or an antigen-binding part thereof which is directed to the surface protein CTLA-4 (anti-CTLA-4), preferably encoding Ipilimumab, an antigen-binding part of Ipilimumab, a functional analog of Ipilimumab, or a functional analog of an antigen-binding part of Ipilimumab. A recombinant NDV strain referred to as “NDV-Mut HN(F277L)-IL-12” is a NDV strain derived from NDV strain MTH-68/H having a mutation in the HN gene at amino acid position 277, wherein the amino acid phenylalanine (F) is substituted to leucine (L) at position 277, and carrying as a foreign gene a gene encoding a protein which improves the cellular immune response and improves the ability of T cells to enter tumor cells, or a part thereof which improves the cellular immune response and improves the ability of T cells to enter tumor cells, preferably encoding interleukin-12 (IL-12), a part of interleukin-12, a functional analog of interleukin-12, or a functional analog of a part of interleukin-12. A recombinant NDV strain referred to as “NDV-Mut HN(F277L)-NS1” is a NDV strain derived from NDV strain MTH-68/H having a mutation in the HN gene at amino acid position 277, wherein the amino acid phenylalanine (F) is substituted to leucine (L) at position 277, and carrying as a foreign gene a gene encoding a protein and/or a part thereof with the ability to modulate the virus replication cycle, preferably encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus. The recombinant NDV strains NDV-Mut HN(F277L)-Ipilimumab, NDV-Mut HN(F277L)-IL-12 and NDV-Mut HN(F277L)-NS1 are provided by the present invention in a preferred embodiment.

Within the meaning of the present invention the term “Newcastle disease virus” or “NDV” includes all known velogenic, mesogenic and lentogenic NDV strains. According to the present invention mesogenic and lentogenic strains are preferred. Still more preferred is the NDV parent strain the NDV strain MTH-68/H (SEQ ID No. 1) or a functional analog of the NDV strain MTH-68/H. The recombinant NDV according to the present invention in this particular preferred embodiment is a NDV derived from NDV strain MTH-68/H.

Within the meaning of the present invention “NDV derived from NDV strain MTH-68/H” is intended to mean that the derived NDV strain is encoded by a viral genome or comprises a viral genome having a nucleic acid sequence with a sequence identity of at least 75%, particularly at least 90%, more particularly at least 95% to the nucleic acid sequence of the NDV strain MTH-68/H. In one embodiment the sequence identity is at least 98%, preferably at least 99% or 100%. Any foreign genes comprised in the recombinant NDV strains are not considered in the sequence alignment, and the reference sequence for this sequence alignment is that of SEQ ID No. 1 of the sequence listing. A sequence variation is for example the substitution, insertion or deletion of at least one nucleotide, preferably the substitution, insertion or deletion of up to 20 nucleotides, still more preferably of up to 15 nucleotides. Any combination of substitution, insertion and deletion is also comprised by that definition, provided that the final construct possesses the desired characteristics. The variation may result in at least one amino acid substitution, wherein each amino acid substitution can be a conservative or a non-conservative amino acid substitution.

It has been surprisingly shown that the mutation in the HN gene results in a beneficially improved replication capability of the respective strain as compared to an otherwise identical NDV not having said mutation in the HN gene. The beneficially increased replication rate of the NDV not only results in higher number of virus particles capable of infecting and destroying cancer cells, but also results in a higher expression of the foreign gene carried by the said recombinant NDV strains. As explained earlier, the respective gene products of the foreign gene results in an improved immunological response and thus an improved destruction of cancer cells.

In a preferred embodiment of the present invention the recombinant and mutated NDV strains comprise in addition to the above described mutation in the HN gene, a mutation in the M gene and the thus encoded matrix protein. Still more preferred the mutated M gene encodes a matrix protein with an amino acid substitution at position 165 to an amino acid with an aromatic side chain, wherein still more preferred the amino acid glycine (G) at position 165 of the M gene is substituted to the amino acid tryptophane (W).

NDV strains comprising both a mutation in the HN gene and the M gene, as described supra, have been shown as being particularly suitable to infect cancer cells and to provide an enhanced replication rate within the cancer cells. Thus, according to one embodiment of the present invention a recombinant NDV strain comprises as a foreign gene at least one gene selected from a gene encoding an antibody directed to the surface protein CTLA-4 or an antigen-binding part directed to the surface protein CTLA-4 (anti-CTLA-4), preferably a gene encoding Ipilimumab, an antigen-binding part or a functional analog of Ipilimumab or its antigen-binding part, a gene encoding a protein which improves the cellular immune response and improves the ability of T cells to enter tumor cells, or a part thereof which improves the cellular immune response and improves the ability of T cells to enter tumor cells, preferably a gene encoding interleukin-12 (IL-12), a part of interleukin-12 or a functional analog of interleukin-12 or a part thereof, a gene encoding a protein with the ability to modulate the virus replication cycle, or a part thereof with the ability to modulate the virus replication cycle, preferably a gene encoding the non-structural protein NS1 of influenza A virus, a part of the non-structural protein NS1 of influenza A virus or a functional analog of the non-structural protein NS1 of influenza A virus or a part thereof, and any combination of these genes or parts or functional analogs thereof, and has a mutation both in the HN gene, preferably at position 277 resulting in a substitution of the amino acid phenylalanine (F) to leucine (L) at amino acid position 277 of the HN gene, and a mutation in the M gene, preferably at amino acid position 165 resulting in a substitution of the amino acid glycine (G) to the amino acid tryptophane (W) at amino acid position 165 of the M gene. As explained supra, other substitutions at these two amino acid positions are also comprised within the present invention.

A NDV strain derived from NDV strain MTH-68/H having a mutation in the HN gene, wherein the amino acid phenylalanine (F) is substituted to leucine (L) at amino acid position 277, and having a mutation in the M gene, wherein the amino acid glycine (G) is substituted to the amino acid tryptophane (W) at amino acid position 165 of the M gene is herein referred to also as NDV-Mut HN(F277L)/M(G165W) or MutHu or NDV-NoThaBene-1. The foreign gene or part or functional analog thereof carried by this strain is referred to as Ipilimumab, IL-12 or NS1. Within the shortcut for the recombinant NDV strains a reference to Ipilimumab is intended to comprise the complete antibody, antigen-bindings parts and functional analogs of the antibody or functional analogs of an antigen-binding part thereof, but also comprise the more generic form as described herein, i.e. an antibody directed to the surface protein CTLA-4 or an antigen-binding part of this antibody. The same applies for the other foreign genes. That is a recombinant NDV strain referred to as “NDV-Mut HN(F277L)/M(G165W)-Ipilimumab” is a NDV strain derived from NDV strain MTH-68/H having a mutation in the HN gene at amino acid position 277, wherein the amino acid phenylalanine (F) is substituted to leucine (L) at position 277, and having a mutation in the M gene, wherein the amino acid glycine (G) is substituted to the amino acid tryptophane (W) at amino acid position 165 of the M gene, and carrying as a foreign gene a gene encoding an antibody is directed to the surface protein CTLA-4 or an antigen-binding part thereof which is directed to the surface protein CTLA-4 (anti-CTLA-4), preferably encoding Ipilimumab, an antigen-binding part of Ipilimumab, a functional analog of Ipilimumab, or a functional analog of an antigen-binding part of Ipilimumab. A recombinant NDV strain referred to as “NDV-Mut HN(F277L)/M(G165W)-IL-12” is a NDV strain derived from NDV strain MTH-68/H having a mutation in the HN gene at amino acid position 277, wherein the amino acid phenylalanine (F) is substituted to leucine (L) at position 277, and having a mutation in the M gene, wherein the amino acid glycine (G) is substituted to the amino acid tryptophane (W) at amino acid position 165 of the M gene, and carrying as a foreign gene a gene encoding a protein which improves the cellular immune response and improves the ability of T cells to enter tumor cells, or a part thereof which improves the cellular immune response and improves the ability of T cells to enter tumor cell, preferably encoding interleukin-12 (IL-12), a part of interleukin-12, a functional analog of interleukin-12, or a functional analog of a part of interleukin-12. A recombinant NDV strain referred to as “NDV-Mut HN(F277L)/M(G165W)-NS1” is a NDV strain derived from NDV strain MTH-68/H having a mutation in the HN gene at amino acid position 277, wherein the amino acid phenylalanine (F) is substituted to leucine (L) at position 277, and having a mutation in the M gene, wherein the amino acid glycine (G) is substituted to the amino acid tryptophane (W) at amino acid position 165 of the M gene, and carrying as a foreign gene a gene encoding a protein and/or a part thereof with the ability to modulate the virus replication cycle, preferably encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus. The NDV strains NDV-Mut HN(F277L)/M(G165W)-Ipilimumab, NDV-Mut HN(F277L)/M(G165W)-IL-12 and NDV-Mut HN(F277L)/M(G165W)-NS1 are provided by the present invention in a preferred embodiment.

According to another embodiment of the present invention, in addition to the mutation at amino acid position 277 of the HN gene and preferably the mutation at amino acid position 165 of the M gene as specified supra, a recombinant NDV according to the present invention may also comprise a mutation in the F gene, which mutation is a mutation which encodes a fusion protein having an amino acid substitution at position 289, and which mutation is capable of improving the oncolytic potential of the NDV strain. Still more preferred, the amino acid at position 289 of the F gene is substituted from leucine (L) to alanine (A). Alternatively or in addition a recombinant NDV according to the present invention may comprise a mutation in the RNA-editing sequence of the P gene. More preferably, the mutation abolishes and/or decreases the expression of the V protein.

Table 1a shows a list of mutations for obtaining NDV variants according to the present invention. The mutation in the HN gene is comprised in all NDV strains according to the present invention, while the mentioned mutations in the M gene and in the F gene at amino acid position 289 may or may not be present. If present, any possible combination of these mutations is encompassed by the present invention.

TABLE 1a

| Gene | Relevant amino acid |
|---|---|
| HN | F277L |
| M | G165W |
| F | L289A |

Table 1b shows particularly preferred NDV variants with increased titer production, and the parent NDV strain MTH-68/H. “+” means that at the mentioned amino acid position the original (first mentioned) amino acid is substituted by the last mentioned amino acid, while “−” means that there is no amino acid substitution at the mentioned position, i.e. the first mentioned amino acid is present. For example and with regard to the HN gene “+” means that at amino acid position 277 phenylalanine (F) is substituted to leucine (L).

TABLE 1b

| Gene | Relevant amino acid | MTH-68/H | | | | |
|---|---|---|---|---|---|---|
| HN | F277L | − | + | + | + | + |
| M | G165W | − | − | + | − | + |
| F | L289A | − | − | − | + | + |

The NDV variants mentioned in tables 1a and 1b carry as a foreign gene at least one gene selected from the group consisting of a gene encoding an antibody directed to the surface protein CTLA-4 or an antigen-binding part directed to protein CTLA-4 (anti-CTLA-4), preferably a gene encoding Ipilimumab, an antigen-binding part or a functional analog of Ipilimumab or its antigen-binding part, a gene encoding a protein which improves the cellular immune response and the ability of T cells to enter tumor cells, or a part thereof which improves the cellular immune response and the ability of T cells to enter tumor cells, preferably a gene encoding interleukin-12 (IL-12), a part of interleukin-12 or a functional analog of interleukin-12 or a part thereof, a gene encoding a protein with the ability to modulate the virus replication cycle, or a part thereof with the ability to modulate the virus replication cycle, preferably a gene encoding the non-structural protein NS1 of influenza A virus, a part of the non-structural protein NS1 of influenza A virus or a functional analog of the non-structural protein NS1 of influenza A virus or a part thereof, and any combination thereof.

In describing a protein or peptide formulation, structure and function herein, reference is made to amino acids. In the present specification, amino acid residues are expressed by using the following abbreviations. Also, unless explicitly otherwise indicated, the amino acid sequences of peptides and proteins are identified from N-terminal to C-terminal (left terminal to right terminal), the N-terminal being identified as a first residue. Amino acids are designated by their 3-letter abbreviation, 1-letter abbreviation, or full name, as follows. Ala: A: alanine; Asp: D: aspartic acid; Glu: E: glutamic acid; Phe: F: phenylalanine; Gly: G: glycine; His: H: histidine; Ile: I: isoleucine; Lys: K: lysine; Leu: L: leucine; Met: M: methionine; Asn: N: asparagine; Pro: P: proline; Gln: Q: glutamine; Arg: R: arginine; Ser: S: serine; Thr: T: threonine; Val: V: valine; Trp: W: tryptophan; Tyr: Y: tyrosine; Cys: C: cysteine.

As used herein, amino acids with a hydrophobic side chain comprise in particular Ala: A: alanine, Ile: I: isoleucine, Leu: L: leucine, Met: M: methionine, Pro: P: proline, and Val: V: valine. Amino acids with an aromatic side chain comprise in particular Phe: F: phenylalanine, Trp: W: tryptophan, and Tyr: Y: tyrosine.

In a preferred embodiment the NDV and the recombinant NDV resulting thereof is encoded by and/or comprises at least one of the nucleic acids according to SEQ ID No. 1 to 5 or parts thereof. According to another preferred embodiment the NDV strains and the recombinant NDV strains of the present invention have a sequence identity of at least 75%, particularly of at least 90%, more particularly of at least 95% to any one of SEQ ID No. 1 to 5. The sequences given by these SEQ ID numbers are as follows:
SEQ ID No. 1: Nucleic acid (cDNA) sequence of the Newcastle disease virus (NDV) strain MTH-68/H genome;
SEQ ID No. 2: Nucleic acid (cDNA) sequence of Newcastle disease virus strain Mut HN(F277L)/M(G165W) genome having a mutation in the HN gene and in the M gene (=NDV-NoThaBene-1 genome);
SEQ ID No. 3: Nucleic acid (cDNA) sequence of Newcastle disease virus strain Mut HN(F277L)/M(G165W)-Ipilimumab genome created by reverse genetics as disclosed herein, wherein the nucleic acid comprises a mutation in the HN gene and in the M gene, and additionally encodes Ipilimumab;
SEQ ID No. 4: Nucleic acid (cDNA) sequence of Newcastle disease virus strain Mut HN(F277L)/M(G165W)-IL-12 genome created by reverse genetics as disclosed herein, wherein the nucleic acid comprises a mutation in the HN gene and in the M gene, and additionally encodes interleukine-12;
SEQ ID No. 5: Nucleic acid (cDNA) sequence of Newcastle disease virus strain Mut HN(F277L)/M(G165W)-NS1 genome created by reverse genetics as disclosed herein, wherein the nucleic acid comprises a mutation in the HN gene and in the M gene, and additionally encodes the non-structural protein NS1 of influenza A virus.

A person skilled in the art is well aware of the fact that the nucleobase thymine in the nucleic acid sequence of DNA is replaced by the nucleobase uracil in the nucleic acid sequence of RNA.

That is in a preferred embodiment of the present invention the viral HN gene product preferably has an amino acid sequence as identified in SEQ ID No. 6, and/or the viral M gene product preferably has an amino acid sequence as identified in SEQ ID No. 7. Also encompassed by the present invention are recombinant NDV strains comprising functional analogs of the gene products according to SEQ ID No. 6 or SEQ ID No. 7.

In another aspect the present invention also provides a recombinant nucleic acid comprising the nucleic acid sequence of the recombinant NDV strains of the present invention.

In describing nucleic acid formulation, structure and function herein, reference is made to any of a group of polymeric nucleotides such as DNA or RNA. Nucleic acid is composed of either one or two chains of repeating units called nucleotides, which nucleotides consist of a nitrogen base (a purine or pyrimidine) attached to a sugar phosphate. In the present specification, nucleotide residues are identified by using the following abbreviations. Adenine residue: A; guanine residue: G; thymine residue: T; cytosine residue: C; uracil residue: U. Also, unless explicitly otherwise indicated, the nucleotide sequences of nucleic acid are identified from 5′-terminal to 3′-terminal (left to right terminal), the 5′-terminal being identified as a first residue.

A nucleic acid according to the present invention comprises a transgenic construct. According to one embodiment the transgenic construct encodes an antibody and/or an antigen-binding part thereof capable of blocking checkpoint inhibition. The antibody or the antigen-binding part is directed to the surface protein CTLA-4 (anti-CTLA-4). According to the present invention the encoded antibody or antigen-binding part thereof is in a particularly preferred embodiment Ipilimumab, an antigen-binding part of Ipilimumab, a functional analog of Ipilimumab or a functional analog of an antigen-binding part of Ipilimumab. According to another embodiment the transgenic construct encodes a protein which improves the cellular immune response and improves the ability of T cells to enter tumor cells, or a part thereof which improves the cellular immune response and improves the ability of T cells to enter tumor cells, preferably interleukin-12 (IL-12), a part of interleukin-12, a functional analog of interleukin-12 or a functional analog of a part of interleukin-12. According to another embodiment of the present invention the transgenic construct encodes a protein with the ability to modulate the virus replication cycle, or a part thereof with the ability to modulate the virus replication cycle. Preferably this encoded protein is the non-structural protein NS1 of influenza A virus, a part of the non-structural protein NS1 of influenza A virus, a functional analog of the non-structural protein NS1 of influenza A virus or a functional analog of a part of the non-structural protein NS1 of influenza A virus.

According to a further embodiment of the present invention, the transgenic construct comprised in the nucleic acid may be a combination of two or more of the foreign genes and/or parts or functional analogs thereof as mentioned supra.

In addition to the transgenic construct a nucleic acid according to the present invention comprises a mutation in the HN gene, said mutation allowing replication of the NDV in a cancer cell to a higher level than replication of an otherwise identical NDV not having the said mutation in the HN gene.

In a preferred embodiment of the present invention the sequence encoding the recombinant NDV comprises a mutated HN gene encoding hemagglutinin-neuramidase with an amino acid substitution at position 277 to an amino acid with a hydrophobic side chain other than phenylalanine. Still more preferred the mutated HN gene encodes for a hemagglutinin-neuramidase referred to as HN^{F277L}. That is the amino acid phenylalanine (F) at position 277 of the hemagglutinin-neuramidase is substituted to leucine (L).

In a particular preferred embodiment the nucleic acid is derived from the NDV strain MTH-68/H.

In another embodiment of the present invention the sequence encoding the recombinant NDV also comprises a mutation in the M gene. Preferably the encoded mutated M gene encodes a matrix protein with an amino acid substitution at position 165 to an amino acid with an aromatic side chain. Most preferred the mutated M gene encodes M^{G165W}. That is the amino acid glycine (G) at position 165 of the M gene is substituted to the amino acid tryptophane (W) at the respective position. In one embodiment of the present invention the mutation in the M gene is comprised in combination with the mutation in the HN gene as specified supra.

In a particular preferred embodiment the nucleic acid is derived from the NDV strain MTH-68/H and comprises a mutation in the HN gene resulting in an amino acid with a hydrophobic side chain other than phenylalanine at position 277, and comprising a mutation in the M gene resulting in a matrix protein with an amino acid substitution at position 165 to an amino acid with an aromatic side chain. Still more preferred the mutated HN gene encodes for a hemagglutinin-neuramidase in which the amino acid phenylalanine (F) at position 277 of the hemagglutinin-neuramidase is substituted to leucine (L), and the mutated M gene encodes for a matrix protein in which the amino acid glycine (G) at position 165 is substituted to the amino acid tryptophane (W).

Also with the scope of the present invention are those nucleic acids comprising, in addition to the mutation in the HN gene, and preferably the M gene, as specified supra, a mutation in the F gene resulting in an amino acid substitution at position 289 of the amino acid sequence and/or a mutation in the P gene as specified supra.

In one embodiment of the present invention the nucleic acid consists of or comprises at least one of the nucleic acids according to SEQ ID No. 2 to 5 or parts thereof. In another preferred embodiment the nucleic acid of the present invention has a sequence identity of at least 75%, particularly of at least 90%, more particularly of at least 95% to any one of the sequences according to SEQ ID No. 2 to 5.

In one embodiment of the present invention the nucleic acid is derived from a recombinant NDV as described supra.

The skilled artisan knows how to use the disclosed sequences to produce synthetic DNAs (e.g. vectors, in particular in rg-NDV vectors), and how to use the synthetic DNAs to reconstruct and express the viral genome. Thus, the skilled artisan is enabled to produce virus particles by using the information disclosed herein.

Recombinant viral genomes, which can be used to rescue virus particles, can for example be obtained by a method called ‘reverse genetics’. Therefore, in another aspect the present invention also provides a method (herein also called ‘reverse genetics’) for providing cloned full-length cDNA of the recombinant NDV strains according to the present invention, and, in a further embodiment, the invention also provides infectious virus particles obtained from said full-length cDNA of the recombinant NDV strains according to the present invention. These recombinant NDV strains have an unexpected beneficial replication capability in cell cultures, eggs or animals that are used to propagate the virus for pharmaceutical purposes, giving production advantages to such improved rgNDV strains. Furthermore, the higher replication rate is also particularly useful to obtain the desired oncolytic effects in vivo in that more virus particle will be produced for subsequent rounds of infection of cancer cells that were missed in the first round of infection. What is more, higher levels (yields) of the foreign gene or the foreign genes encoded by the transgenic construct will be achieved for providing the desired enhanced oncolytic effects in vivo. Thus, by modifying the HN protein within a transgene-expressing NDV higher yields of the transgenic construct protein can be obtained, which is very useful in anti-tumor immunotherapy.

The recombinant NDV strains according to the present invention can be obtained by a reverse genetic method for preparing an rgNDV encoding at least one foreign gene and having improved replication in a cancer cell over a parent NDV.

The method according to the present invention comprising providing a nucleic acid construct encoding a HN gene with a mutation, particularly wherein the mutation in the HN gene leads to a change in the expression of the hemagglutinin-neuraminidase, wherein the amino acid, particularly phenylalanine (F), in position 277 is substituted, particularly wherein the amino acid in position 277 is substituted to an amino acid with a hydrophobic side chain, more particularly wherein the amino acid in position 277 is substituted to leucine (L) at amino acid position 277 of the HN gene.

The method further comprises a step of providing a nucleic acid encoding a rgNDV further comprising a transgenic construct encoding an antibody directed to the surface protein CTLA-4 or an antigen-binding part directed to the surface protein CTLA-4 (anti-CTLA-4), preferably encoding Ipilimumab, an antigen-binding part or a functional analog of Ipilimumab or its antigen-binding part, and/or a protein which improves the cellular immune response and improves the ability of T cells to enter tumor cells, or a part thereof which improves the cellular immune response and improves the ability of T cells to enter tumor cells, preferably a gene encoding interleukin-12 (IL-12), a part of interleukin-12, a functional analog of interleukin-12, or a functional analog of a part of interleukin-12, and/or a protein with the ability to modulate the virus replication cycle, or a part thereof with the ability to modulate the virus replication cycle, preferably a gene encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus.

In the next step of the claimed method the nucleic acid construct with said mutation in the HN gene is incorporated in the nucleic acid encoding a rgNDV comprising the transgenic construct in order to obtain a full length cDNA of the NDV, which additionally comprises a transgenic construct as specified supra. In a preferred embodiment this method step results in a nucleic acid as disclosed supra.

The nucleic acid encoding a rgNDV further comprising a transgenic construct as specified supra can be obtained by generating sub-genomic cDNA fragments and assembling the full length cDNA of the recombinant NDV from these fragments.

Following this method step the thus obtained nucleic acid encoding a recombinant and mutated rgNDV according to the present invention is used to produce infectious rgNDV particles, which replication characteristics and expression rates of the encoded foreign protein(s) in cancer cells are compared with the replication characteristics and expressions rates of the parent NDV used for designing the recombinant and mutated NDV strain.

Finally, said rgNDV is selected for further use, when it shows both an improved replication characteristic over the parent NDV and a sufficient expression of the gene product or gene products of the foreign genes.

If it is intended to introduce further to the mutation in the HN gene, a further mutation in a viral gene, preferably the M gene, the F gene and/or the P gene, the selected recombinant and mutated rgNDV comprising a transgenic construct as specified supra and a mutated HN gene is used for incorporating a nucleic acid construct encoding a mutated M gene, F gene or P gene, and the method for introducing the mutation in the HN gene is repeated respectively. Alternatively there is already provided a nucleic acid encoding a rgNDV with the transgenic construct and carrying a mutation in the M gene, F gene or P gene, particularly wherein said mutation is capable of improving oncolytic potential of said rgNDV.

According to a preferred embodiment of the present invention the mutation in the M gene comprises or is a mutation which encodes a matrix protein with an amino acid substitution at position 165 to an amino acid with an aromatic side chain. Still more preferred is a substitution of the amino acid glycine (G) at amino acid position 165 of the M gene to the amino acid tryptophane (W).

According to another preferred embodiment of the present invention the mutation in the F gene comprises or is a mutation which encodes a fusion protein having an amino acid substitution in position 289, which mutation is capable of improving the oncolytic potential of the NDV strain. Still more preferred, the amino acid at position 289 is substituted from leucine (L) to alanine (A).

According to another preferred embodiment of the present invention the mutation is in the RNA-editing sequence of the P gene. More preferably, the mutation abolishes and/or decreases the expression of the V protein.

It is also within the scope of the present invention that the viral genome comprises a combination of two or more mutations, which are selected from a mutation in the HN gene, a mutation in the M gene, a mutation in the F gene and a mutation in the P gene. For the details of the respective mutations, reference to detailed disclosure of the each of these mutations given supra is made.

The invention also provides an isolated, recombinant nucleic acid encoding a rgNDV obtainable by a method for preparing an rgNDV having improved replication in a cancer cell in comparison to that of a parent NDV, as described supra.

In one preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene in combination with a transgenic construct encoding an antibody directed to the surface protein CTLA-4 or an antigen-binding part directed to the surface protein CTLA-4 (anti-CTLA-4), preferably encoding Ipilimumab, an antigen-binding part of Ipilimumab, a functional analog of Ipilimumab or a functional analog of an antigen-binding part of Ipilimumab. The rgNDV may further comprise at least one mutation selected from a mutation in the M gene, preferably at amino acid position 165, still more preferably G165W, a mutation at amino acid position 289 in the F gene, preferably L289A, and a mutation in the P gene, preferably the mutation abolishes and/or decreases the expression of the V protein.

In another much preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (an F277L mutation) allowing replication of said rgNDV in a human cancer cell to a higher level than replication of an otherwise identical rgNDV having the amino acid phenylalanine (F) at position 277 in the HN gene in combination with a transgenic construct encoding a protein which improves the cellular immune response and improves the ability of T cells to enter tumor cells, or a part thereof which improves the cellular immune response and improves the ability of T cells to enter tumor cells, preferably interleukin-12 (IL-12), a part of interleukin-12, a functional analog of interleukin-12, or a functional analog of a part of interleukin-12. The rgNDV may further comprise at least one mutation selected from a mutation in the M gene, preferably at amino acid position 165, more preferably G165W, a mutation at position 289 in the F gene, preferably L289A, and a mutation in the P gene, preferably the mutation abolishes and/or decreases the expression of the V protein.

In another much preferred embodiment the isolated and recombinant nucleic acid encodes a rgNDV provided with a mutation in the HN gene resulting in a change of the amino acid phenylalanine (F) at amino acid position 277 of the hemagglutinin-neuraminidase into a leucine (L), said mutation (a F277L mutation) allowing replication of said rgNDV in a human cancer cell to a higher level than replication of an otherwise identical rgNDV having the amino acid phenylalanine (F) at position 277 in the HN gene in combination with a transgenic construct encoding a protein with the ability to modulate the virus replication cycle, or a part thereof with the ability to modulate the virus replication cycle, preferably encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus. The rgNDV may further comprise at least one mutation selected from a mutation in the M gene, preferably at amino acid position 165, more preferably G165W, a mutation at position 289 in the F gene, preferably L289A, and a mutation in the P gene, preferably the mutation abolishes and/or decreases the expression of the V protein.

According to a particularly preferred embodiment the nucleic acid used to engineer a recombinant NDV genome is derived from the NDV strain MTH-68/H. Thus the present invention provides in one embodiment nucleic acids, preferably obtainable by the above specified method, which are
rgNDV-Mut HN(F277L)-Ipilimumab,
rgNDV-Mut HN(F277L)-IL-12,
rgNDV-Mut HN(F277L)-NS1.

In another preferred embodiment the present invention provides nucleic acids, preferably obtainable by the above specified method, which are
rgNDV-Mut HN(F277L)/M(G165W)-Ipilimumab (=rgNDV-NoThaBene-1-Ipilimumab),
rgNDV-Mut HN(F277L)/M(G165W)-IL-12 (=rgNDV-NoThaBene-1-IL-12),
rgNDV-Mut HN(F277L)/M(G165W)-NS1 (=rgNDV-NoThaBene-1-NS1).

A NDV or rgNDV according to the present invention can be provided in a suitable cell or cell line, for example a HeLa cell line or in cancer cells, or an embryonated egg that is susceptible to a NDV infection.

In these infected cells the virus is grown to sufficient quantities, and preferably under sufficient conditions such that the virus is free from exogenous contamination, and the progeny virus particles are collected by suitable methods well known to those skilled in the art.

In a further aspect the present invention is concerned with the medical use of the recombinant NDV strains (the virus particles) of the present invention. In this regard the invention provides an oncolytic NDV for use in oncological treatment (this term is used herein also replaceable with “for use as a medicament, especially in a method of treatment of cancer”; or the use of said oncolytic NDV in the preparation of a pharmaceutical formulation for use in said method of treatment) in humans (or more generically animals, such as mammalian animals).

In this regard the present invention is first concerned with a recombinant NDV according to the present invention for use in medicine. Secondly, the present invention is more particularly concerned with a recombinant NDV according to the present invention for use in a method of treating cancer in a subject considered in need thereof.

In particular one or more of the recombinant and mutated NDV strains of the present invention are used in a method for treating one or more indications selected from the group consisting of brain tumors, like glioblastoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.

In a preferred embodiment of the present invention the subject to be treated is a mammal, particularly a mammalian animal or a human subject. Still more preferred the one or more oncolytic NDV strains of the present invention may be used for the treatment of adults and/or children, preferably human adults and/or human children.

In one embodiment only one recombinant and mutated NDV or rgNDV strain according to the present invention is used in a method for treating cancer, in particular a cancer selected from the specific cancers as described and/or mentioned in the claims herein. Alternatively in another preferred embodiment of the present invention a combination of at least two, preferably of at least three, of the recombinant and mutated NDV or rgNDV strains according to the present invention is used in a method for treating cancer, in particular a cancer selected from the specific cancers as described and/or mentioned in the claims herein. In particular it is preferred to use a combination of virus particles, wherein each virus particle encodes and expresses another foreign gene selected from the group consisting of a gene encoding an antibody or an antigen-binding part thereof both directed to the surface protein CTLA-4 (anti-CTLA-4), preferably encoding Ipilimumab, a part of Ipilimumab, a functional analog of Ipilimumab, or a functional analog of a part of Ipilimumab, a gene encoding a protein which improves the cellular immune response and improves the ability of T cells to enter tumor cells, or a part thereof which improves the cellular immune response and improves the ability of T cells to enter tumor cells, preferably encoding interleukin-12 (IL-12), a part of interleukin-12, a functional analog of interleukin-12, or a functional analog of a part of interleukin-12, a gene encoding a protein with the ability to modulate the virus replication cycle, or a part thereof with the ability to modulate the virus replication cycle, preferably encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus. The use of such a combination of foreign genes results in an active combination of proteins beneficially improving the immunological response with the cancer.

Also within the scope of the present invention is the use of one or more NDV or rgNDV strains according to the present invention in combination with one or more other therapies suitable for the treatment of cancer. In a preferred embodiment the one or more NDV or rgNDV strains or a pharmaceutical formulation comprising the same is used in a method for treating cancer, especially a cancer selected from the specific cancers as described and/or mentioned in the claims herein.

The one or more NDV or rgNDV strains and one or more other therapies can be used concurrently or sequentially. In certain embodiments, the one or more NDV or rgNDV and the one or more other therapies are administered in the same (“fixed”) pharmaceutical formulation. In other embodiments, the one or more NDV or rgNDV strains and the one or more other therapies are administered in different formulations. The one or more NDV or rgNDV strains of the present invention and the one or more other therapies can be administered by the same or different routes of administration to the subject considered in need thereof. Another therapy within the meaning of the present invention also comprises an administration of other oncolytical virus strains, for example recombinant NDV or rgNDV strains, in particular recombinant NDV or rgNDV strains derived from NDV strain MTH-68/H, encoding and expressing foreign genes other than those as disclosed within the present invention. Another foreign gene expressed by the NDV or rgNDV, in particular the recombinant NDV or rgNDV strains derived from NDV strain MTH-68/H, may be
a gene encoding a protein with the ability to selectively induce apoptosis in human tumor cells, but not in normal human cells, or a part thereof with the ability to selectively induce apoptosis in human tumor cells, but not in normal human cells, preferably a gene encoding Apoptin (VP3 from chicken anemia virus), a part of Apoptin, a functional analog of Apoptin or a functional analog of a part of Apoptin,
a gene encoding a protein that reduces or inhibits IFN expression such as an IFN-beta receptor, or a part thereof that reduces or inhibits IFN expression such as an IFN-beta receptor, preferably a gene encoding the viral protein B18R from vaccinia virus, a part of the viral protein B18R from vaccinia virus or a functional analog of the viral protein B18R from vaccinia virus or a functional analog of a part of the viral protein B18R, and/or
a gene encoding an antibody directed to protein PD-1 or an antigen-binding part directed to protein PD-1 (anti-PD-1), preferably a gene encoding Nivolumab, an antigen-binding part of Nivolumab, a functional analog of Nivolumab, or a functional analog of an antigen-binding part of Nivolumab.

In a preferred embodiment the foreign gene expressed by the recombinant NDV or rgNDV is Apoptin, B18R and/or Nivolumab.

Particularly preferred NDV strains and rgNDV which may be used in combination are disclosed in the pending European patent application no. 18166400.4. For details of these strains reference to these patent applications is made.

In a further aspect the present invention also provides a pharmaceutical formulation comprising a recombinant and mutated NDV strain, preferably an rgNDV, as described herein, without or preferably with at least one pharmaceutically acceptable carrier material.

For the indications mentioned herein, the appropriate dosage will, of course, vary depending upon, for example, the particular molecule of the invention to be employed, the mode of administration and the nature and severity of the condition being treated. In general, the dosage, in one embodiment of the invention, can preferably be in the range of 10⁷ to 10⁹ pfu per dose.

The recombinant and mutated NDV strains according to the present invention provide on the one hand an improved replication capacity of the NDV particles in cancer cells, which is associated with increased cancer cell lysis and increased anti-cancer activity, because the recombinant and mutated NDV strains of the present invention are still selective for cancer cells. The low to sero activity towards normal cells is associated with an increased therapeutic window or safety margin, as is commonly determined for cancer therapeutics. Beside the virological activity the recombinant mutated NDV strains of the present invention are in addition beneficial for the treatment of cancer from an immunological point of view. This is because the foreign gene carried by NDV strains of the present invention beneficially influences the ability of the immune system of the subject to be treated to attack and destroy cancer cells. Advantageously the foreign gene is expressed inside the tumor to be treated, that is directly in the side of need.

The pharmaceutical formulations or pharmaceutical compositions of the present invention may be manufactured in any conventional manner, which are known to those skilled in the art. For example a pharmaceutical composition according to the present invention comprising a recombinant virus particle as described within this description can be provided in a dried, preferably in a lyophilized form and can be complemented with a suitable solvent, e.g. an aqueous carrier for injection at the time when used for administration. The dried form is very stable. Thus, in one embodiment the pharmaceutical composition is present as a dried form, still more preferred in a lyophilized form, and is reconstituted before administration by adding a suitable solvent. That is the present invention also relates to the above described dried form of the pharmaceutical composition for use as a medicament, preferably for use in a method of treating cancer. The process parameters for drying, preferably lyophilisation must be chosen such that the dried/lyophilized product contains live, replication competent viral particles, which can be grown to standardized titers, when needed. A person skilled in the art knows how to set these parameters.

For administration of a dried form, the dried form is directly dissolved in a suitable solvent or pharmaceutically acceptable carrier for injection and/or stabilization of the NDV or rg NDV and/or for improving its delivery to cancer cells. The solvent or carrier is preferably an aqueous carrier, for example sterile water for injection or sterile buffered physiological saline or another, preferably isotonic buffer system having a pH in the range of 7.2 to 7.6. The reconstitution can be performed ideally at or close to the intended time of administration in order to avoid any contaminations with microbes, etc. The skilled person is familiar with the handling of pharmaceutical compositions for reconstitution and reconstituted solutions.

In another embodiment, the present invention is concerned with pharmaceutical formulations comprising cancer cells infected with a transgene-expressing NDV or transgene-expressing rgNDV strain as described herein, and a pharmaceutically acceptable carrier. In specific embodiments, the cancer cells have been treated with gamma radiation prior to incorporation into the pharmaceutical formulation. In specific embodiments, the cancer cells have been treated with gamma radiation before infection with the NDV strain (e.g., rgNDV). In other specific embodiments, the cancer cells have been treated with gamma radiation after infection with the NDV (e.g. rgNDV). In another embodiment, presented herein are pharmaceutical formulations comprising a protein concentrate from lysed NDV-infected cancer cells (e.g., rgNDV infected cancer cells), and a pharmaceutically acceptable carrier.

The pharmaceutical formulation of the present invention can be prepared by a method comprising or consisting of the steps of:
propagating an NDV, preferably an rgNDV, according to the present invention in at least one cell or embryonated egg that is susceptible to a NDV infection;
collecting the progeny virus particles, wherein the virus is grown to sufficient quantities and under sufficient conditions that the virus is free from exogenous contamination, such that the progeny virus is suitable for formulation into a pharmaceutical formulation; and
preferably manufacturing a pharmaceutical formulation comprising said progeny virus particles in the absence of or after addition of at least one pharmaceutically acceptable carrier material.

Thus in a further aspect the present invention also provides a method for producing the above mentioned pharmaceutical formulation. It is also within the scope of the present invention that for manufacturing the said pharmaceutical formulation a nucleic acid according to the present invention is used, from which virus particles can be rescued which are then propagated in at least one cell or embryonated egg that is susceptible to a NDV infection.

The step of collecting progeny virus particles may in one embodiment also comprise a step of processing the collected virus-containing material to enrich virus particles and/or to eliminate host cell DNA.

In a further aspect the present invention also provides a cell or cell line or an embryonated egg comprising a NDV, preferably a rgNDV, according to the present invention.

In still a further aspect the present invention is also concerned with a method for treating cancer, especially a cancer selected from the specific cancers as described and/or mentioned in the claims herein, in a subject considered in need thereof. The method for treating cancer utilizes a transgene-expressing NDV or transgene-expressing rgNDV described herein or any combination thereof or a pharmaceutical formulation comprising such a NDV or rgNDV or any combination thereof, especially in a therapeutically effective amount. Within the meaning of the present patent application a therapeutically effective amount also includes an effective amount for preventing a cancer, in particular a cancer selected from the specific cancers as described and/or mentioned in the claims herein.

The method of treatment comprises or consists of administering to a subject in need thereof a NDV or an rgNDV according to the present invention or any combination thereof in a sufficient amount for infecting and destroying some or all of the cancer cells. In a specific embodiment the method for treating cancer comprises or consists of infecting a cancer cell in a subject with a NDV or rgNDV of the present invention or any combination thereof or with a pharmaceutical composition of the present invention. In another embodiment the one or more NDV or rgNDV or a pharmaceutical composition comprising the same is administered to a subject in need thereof by intravenous, intra-arterial, intratumoral, intramuscular, intradermal, subcutaneous, or any other medically relevant route of administration. According to another embodiment of the present invention the NDV or rgNDV according to the present invention or any combination thereof is administering to a subject in need thereof by administering cancer cells infected with a NDV or rgNDV according to the present invention, especially in a therapeutically effective amount, or pharmaceutical formulation thereof by intravenous, intra-arterial, intratumoral, intramuscular, intradermal, subcutaneous, or any other medically relevant route of administration. In specific embodiments, the cancer cells have been treated with gamma radiation prior to administration to the subject or incorporation into the pharmaceutical formulation. In still another embodiment, a method for treating cancer comprises or consists of administering to a subject in need thereof protein concentrates or plasma membrane fragments from cancer cells infected with a NDV or rgNDV or a pharmaceutical formulation according to the present invention.

In a particular preferred embodiment the method for treating a cancer, in particular a cancer selected from the specific cancers as described and/or mentioned in the claims herein, comprises administering a mixture of at least two, preferably of at least three, of the recombinant and mutated NDV or rgNDV strains according to the present invention. In particular it is preferred to use a combination of virus particles, wherein each virus particle encodes and expresses another foreign gene selected from the group consisting of a gene encoding an antibody and/or an antigen-binding part thereof both directed to the surface protein CTLA-4 (anti-CTLA-4), preferably encoding Ipilimumab, an antigen-binding part of Ipilimumab, a functional analog of Ipilimumab, or a functional analog of an antigen-binding part of Ipilimumab, a gene encoding a protein which improves the cellular immune response and improves the ability of T cells to enter tumor cells, or a part thereof which improves the cellular immune response and improves the ability of T cells to enter tumor cells, preferably encoding interleukin-12 (IL-12), a part of interleukin-12, a functional analog of interleukin-12, or a functional analog of a part of interleukin-12, a gene encoding a protein with the ability to modulate the virus replication cycle, or a part thereof with the ability to modulate the virus replication cycle, preferably encoding the non-structural protein NS1 of influenza A virus, a part of the non-structural protein NS1 of influenza A virus, a functional analog of the non-structural protein NS1 of influenza A virus, or a functional analog of a part of the non-structural protein NS1 of influenza A virus. The administration of such a combination of foreign genes results in an active combination of proteins beneficially improving the immunological response with the cancer. The administration of this mixture of NDVs or rgNDVs according to the present invention can be achieved as specified supra.

Also within the scope of the present invention is a method of treating cancer, especially a cancer selected from the specific cancers as described and/or mentioned in the claims herein, which utilizes a NDV or rgNDV according to the present invention in combination with one or more other therapies. In a preferred embodiment the NDV or rgNDV or a pharmaceutical formulation comprising the same is administered to a subject in need thereof in a therapeutically effective amount. The NDV or rgNDV and one or more other therapies can be administered concurrently or sequentially to the subject (meaning they are jointly therapeutically active). In certain embodiments, the NDV or rgNDV and one or more other therapies are administered in the same (“fixed”) pharmaceutical formulation. In other embodiments, the NDV or rgNDV and one or more other therapies are administered in different formulations. The NDV or rgNDV of the present invention and one or more other therapies can be administered by the same or different routes of administration to the subject considered in need thereof. Another therapy within the meaning of the present invention also comprises an administration of other oncolytical virus strains, for example recombinant NDV or rgNDV strains encoding and expressing foreign genes other than those as disclosed within the present invention. Another foreign gene expressed by the NDV or rgNDV may be
a gene encoding a protein with the ability to selectively induce apoptosis in human tumor cells, but not in normal human cells, or a part thereof with the ability to selectively induce apoptosis in human tumor cells, but not in normal human cells, preferably a gene encoding Apoptin (VP3 from chicken anemia virus), a part of Apoptin, a functional analog of Apoptin or a functional analog of a part of Apoptin,
a gene encoding a protein that reduces or inhibits IFN expression such as an IFN-beta receptor, or a part thereof that reduces or inhibits IFN expression such as an IFN-beta receptor, preferably a gene encoding the viral protein B18R from vaccinia virus, a part of the viral protein B18R from vaccinia virus or a functional analog of the viral protein B18R from vaccinia virus or a functional analog of a part of the viral protein B18R, and/or
a gene encoding an antibody directed to protein PD-1 or an antigen-binding part directed to protein PD-1 (anti-PD-1), preferably a gene encoding Nivolumab, an antigen-binding part of Nivolumab, a functional analog of Nivolumab, or a functional analog of an antigen-binding part of Nivolumab.

In a preferred embodiment the foreign gene expressed by the NDV or rgNDV is Apoptin, B18R and/or Nivolumab.

Particularly preferred NDV strains and rgNDV which may be used in combination are disclosed in the pending European patent application no. 18166400.4. For details of these strains reference to these patent applications is made.

Another therapy may also comprise or may be radiotherapy for cancer.

### FIGURES

FIG. 1 is a schematic presentation of the NDV reverse genetics system. The upper part shows the composition of the full-length cDNA plasmid which contains the full-length NDV cDNA (encoding the nucleocapsid protein (NP), the phosphoprotein (P), the matrix protein (M), the fusion protein (F), the hemagglutinin-neuraminidase (HN), and the RNA-dependent RNA polymerase (L)) cloned behind the bacteriophage T7 RNA Polymerase promoter (T7P; yellow triangle) and followed by a ribozyme sequence (Rz) and T7 transcription termination signal (T7T).

A suitable host cell (shaded round-cornered box) is infected with a recombinant Fowlpox virus that expresses T7 DNA-dependent RNA polymerase (Fowlpox-T7) and subsequently co-transfected with the full-length cDNA plasmid and three helper plasmids containing the genes encoding the NDV NP, P and L proteins, respectively. Transcription of the full-length cDNA results in the generation of the NDV antigenome RNA which is encapsidated by NP protein then transcribed and replicated by the RNA-leading to the generation of infectious NDV.

FIG. 2A is a schematic presentation of the genome of a rgNDV-Mut HN(F277L)/M(G165W)-Ipilimumab (17,436 bp) according to the present invention. The figure shows the position of the gene Ipilimumab which is inserted as extra transcription unit into the rgNDV-Mut HN(F277L)/M(G165W) genome between the P and M genes.

FIG. 2B is a further schematic presentation of the genomes of rgNDV-mutants according to one embodiment of the present invention. The figure shows the position of the respective foreign gene (Ipilimumab, IL-12 or NS1), which is inserted as extra transcription unit into the rgNDV-mutant genome between the P gene and the M gene.

FIG. 3 shows growth kinetics in HeLa cells. HeLa cells were infected at a multiplicity of infection (MOI) of 0.01 with the indicated viruses. At 0 h, 8 h, 24 h and 48 h after infection, the amount of infectious virus in the supernatant was determined by end-point titration on QM5 cells. MTH68: NDV strain MTH-68/H; MutHu: NDV-Mut HN(F277L)/M(G165W); rgMTH68: NDV strain MTH-68/H derived by reverse genetics from cloned full-length cDNA; rgMutHu: NDV strain NDV-Mut HN(F277L)/M(G165W) derived by reverse genetics from cloned full-length cDNA; rgMutHu(HNL277F): rgMutHu in which the amino acid mutation at position 277 in the HN gene was converted from L back to F; rgMutHu(MW165G): rgMutHu in which the amino acid mutation at position 165 in the M gene was converted from W back to G.

FIG. 4 shows a calibration curve of a human IgG ELISA used for determining and quantifying the expression of Ipilimumab from a recombinant NDV strain according to the present invention.

FIG. 5 shows the expression of non-structural protein 1 by rgMutHu-NS1. The expression of NS1 protein was verified by means of immunological staining using H1N1 NS1 antibody, pAb, Rabbit from GenScript (https://www.genscript.com/antibody/A01551-H1N1 NS1 Antibody pAb Rabbit.html) against influenza A (H1N1) NS1 protein. Briefly, rgMutHu-NS1 infected or uninfected cell monolayers were fixed and the NS1-specific antibody was used in an immune-peroxidase monolayer assay (IPMA). The red-brown colour indicates the presence of the NS1 protein. FIG. 5A shows non-infected cells, FIG. 5B shows cells infected with rgMutHu-NS1. The strain rgMutHu-NS1 is the NDV strain NDV-Mut HN(F277L)/M(G165W) derived by reverse genetics from cloned full-length cDNA carrying as foreign gene non-structural protein NS1.

### EXAMPLES

### Example 1. Nucleotide Sequence Analysis of Mutant NDV-Mut HN(F277L)/M(G165W)

We identified a spontaneous mutant of an oncolytic NDV strain MTH-68/H (Csatary et al., 1999, Anticancer Res. 19:635-638.; further called MTH68). The replication capacity of the mutant strain (designated NDV-Mut HN(F277L)/M(G165W) in a variety of human neoplastic cell lines, as well as autologous primary tumors, is greatly enhanced as compared to the original MTH-68/H strain (also referred to as MTH68 strain). We analyzed its nucleotide sequence and found that, compared to MTH68, NDV-HN(F277L)/M(G165W) has two nucleotide mutations, one leading to an amino acid substitution in the M protein (G165W) and the other in the HN protein (F277L).

### Example 2. A Reverse Genetics System that Allows Genetic Modification of NDV-Strains

### 2.1 Reverse Genetics

In order to be able to genetically modify the genome of an RNA virus such as NDV, a manipulatable genetic system must be developed that uses a copy of the full viral RNA (vRNA) genome in the form of DNA. This full-length cDNA is amenable to genetic modification by using recombinant DNA techniques. The authentic or modified cDNA can be converted back into vRNA in cells, which in the presence of the viral replication proteins results in the production of a new modified infectious virus. Such ‘reverse genetics systems’ have been developed in the last few decades for different classes of RNA viruses. This system enables the rapid and facile introduction of mutations and deletions and the insertion of a transgene transcriptional unit, thereby enabling the changing of the biological properties of the virus.

Reverse genetics systems for several NDV strains, including lentogenic as well as velogenic strains, were developed by the Central Veterinary Institute (CVI), part of Wageningen University and Research, currently Wageningen Bioveterinary Research (WBVR) under the supervision of Dr. Ben Peeters (Peeters et al., 1999, J. Virol. 73:5001-9; de Leeuw et al., 2005, J. Gen. Virol. 86:1759-69; Dortmans et al., 2009, J. Gen. Virol. 90:2746-50). In order to generate a reverse genetics system for providing the NDV nucleic acids and strains according to the present invention, a similar approach was used. Details of the procedure can be found in the above cited papers and in the paragraphs below. Briefly, the system consists of 4 components, i.e., a transcription plasmid containing the full-length (either authentic or genetically modified) cDNA of the virus, which is used to generate the vRNA, and 3 expression plasmids (‘helper plasmids’) containing the NP, P and L genes of NDV respectively, which are used to generate the vRNA-replication complex (consisting of NP, P and L proteins). Transcription of the cDNA (i.e. conversion of the cDNA into vRNA) and expression of the NP, P and L genes by the helper plasmids is driven by a T7 promoter. The corresponding T7 DNA-dependent RNA polymerase (T7-RNAPol) is provided by a helper-virus (Fowlpox-T7).

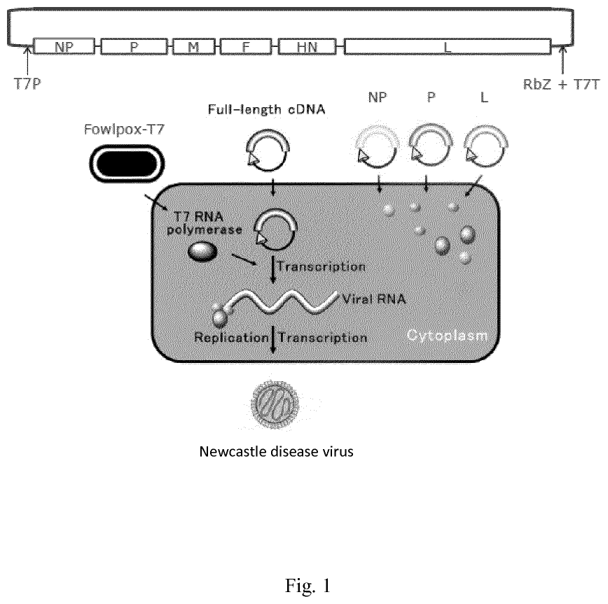

In order to rescue virus, the 4 plasmids are co-transfected into Fowlpox-T7 infected cells (FIG. 1). Three to five days after transfection, the supernatant is inoculated into specific-pathogen-free embryonated chicken eggs (ECE) and incubated for 3 days. Infectious virus that is produced by transfected cells will replicate in the ECE and progeny virus can be harvested from the allantois fluid.

In order to develop a reverse genetics system for NDV the following steps were followed:
Generation of sub-genomic NDV and foreign gene cDNA's by RT-PCR
Assembly of full-length cDNA in a transcription vector
Cloning of each of the NP, P and L genes into an expression vector
Verify nucleotide sequence of full-length cDNA and helper-plasmids
Repair nucleotide differences resulting from the cloning procedure, if necessary
Rescue of infectious virus from cDNA using co-transfection (FIG. 1)
   2.2 Construction of Full-Length NDV-Mut HN(F277L)/M(G165W) cDNA and Helper Plasmids

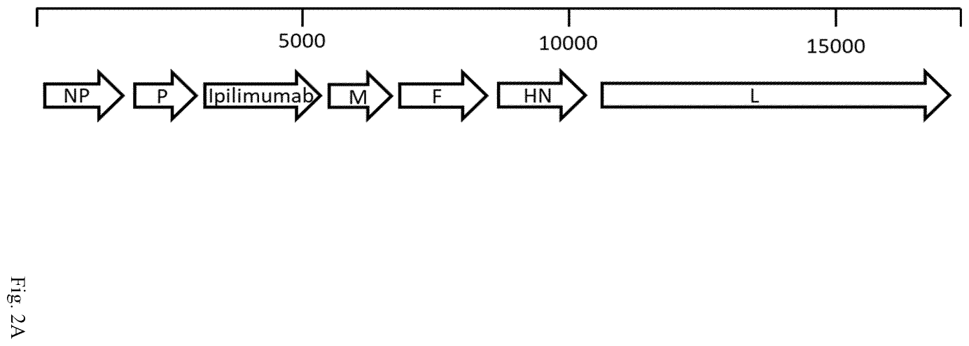

NDV-Mut HN(F277L)/M(G165W) (passage 28 HeLa cells) was used for the isolation of vRNA using standard procedures. The vRNA was used to generate first-strand cDNA by means of Reverse Transcriptase followed by PCR to generate 4 sub-genomic cDNA fragments (designated C1, C2, C3 and C8). The full-length cDNA of NDV-MutHu was assembled from these fragments and cloned in the transcription vector pOLTV5 (Peeters et al., 1999, J. Virol. 73:5001-5009) by a combination of In-Fusion® cloning and classical cloning using restriction enzymes. An overview of the procedure is shown in FIG. 2, and further details can be found in Appendix 1 of this reference. The NP, P and L-genes of NDV-MutHu were obtained by RT-PCR (Appendix 1) and cloned in the expression plasmid pCVI which was derived by deletion of a ClaI restriction fragment from pCI-neo (Promega).

### 2.3 Nucleotide Sequence Analysis

Nucleotide sequence analysis was used to verify that the sequence of pFL-NDV Mut HN(F277L)/M(G165W) was correct. A few nucleotides which differed from the Reference sequence were repaired. Silent mutations (i.e., not leading to an amino acid change) may be left unchanged.

2.4 Rescue of Infectious Virus from pFL-NDV Mut HN(F277L)/M(G165W)

In order to generate infectious virus, we used the co-transfection system described above (and illustrated in FIG. 1) to transfect QM5 cells (derived from Quail) using plasmid pFL-NDV_Mut HN(F277L)/M(G165W) and the helper plasmids pCVI-NP^{Mut HN(F277L)/M(G165W)}, pCVI-P^{Mut HN(F277L)/M(G165W)} and pCVI-L^{Mut HN(F277L)/M(G165W)}. Rescue of infectious virus was successful as demonstrated by the presence of infectious virus in the allantoic fluid of ECE that were inoculated with the transfection supernatant (data not shown). The identity of the rescued virus was determined by RT-PCR followed by sequencing. The rescued virus was designated rgMut HN(F277L)/M(G165W). NDV-Mut HN(F277L)/M(G165W) and rgMut HN(F277L)/M(G165W) have similar growth kinetics in HeLa cells.

### Example 3. Identify Whether One or Both of the Amino Acid Substitutions in Mut HN(F277L)/M(G165W) are Responsible for the Difference in Growth Kinetics Between Mut HN(F277L)/M(G165W) and the Parent Strain MTH68

### 3.1 Growth Kinetics in HeLa Cells

The rescued rg-viruses (Table 1) as well as the original Mut HN(F277L)/M(G165W) and MTH68 viruses were used to determine their growth-kinetics in HeLa cells. Briefly, 4×10⁶ HeLa cells were seeded in 25 cm² cell culture flasks and grown overnight. The cells were infected using a MOI of 0.01 (i.e., 1 infectious virus particle per 100 cells), and at 8, 24 and 48 hours after infection the virus titer in the supernatant was determined by end-point titration on QM5 cells.

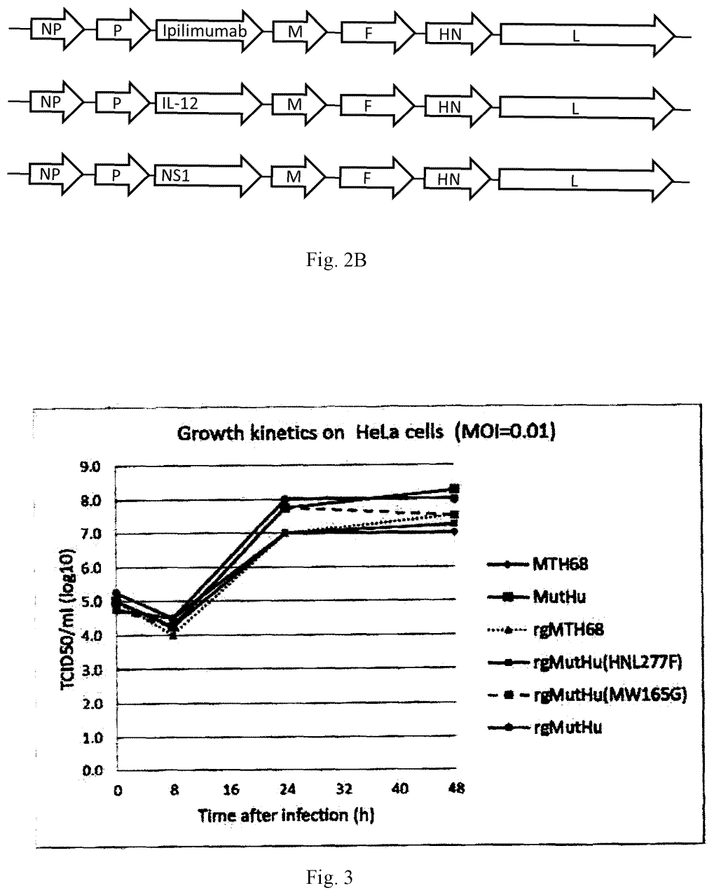

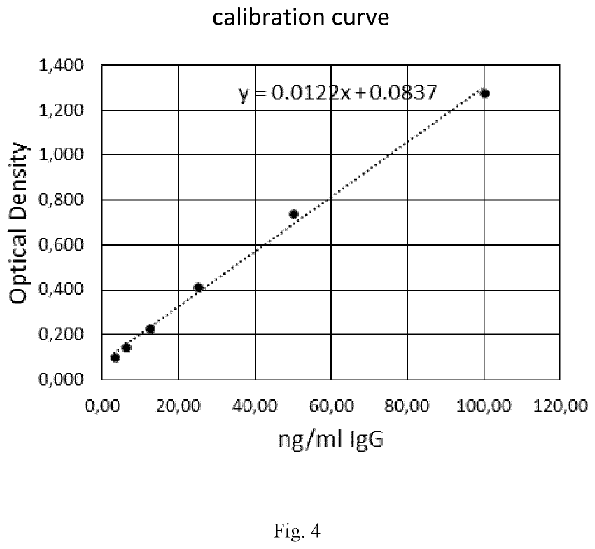

The data (FIG. 3) indicate that strains Mut HN(F277L)/M(G165W) and rgMut HN(F277L)/M(G165W) yield at least 10-fold higher virus titers than MTH68. Furthermore, the data indicate that the mutation at amino acid position 277 in the HN gene is responsible for this effect. The M mutation does not seem to have an effect.

This can be best seen when looking at the virus titers 24 h after infection, or even better when comparing the increase in virus titer between 8 h and 24 h (the exponential growth phase). The virus titer shows an increase of 3.5 (log 10) for Mut HN(F277L)/M(G165W), rgMut HN(F277L)/M(G165W) and rgMut HN(F277L)/M(W165G), whereas this is 2.5 for MTH68, 2.7 for rgMut HN(L277F) and 3.0 for rgMTH68 (Table 3).

rgMut HN(F277L)/M(W165G) is a strain in which the mutation in the M gene has been restored in accordance with the NDV MTH-68/H.

TABLE 1
Virus titers (log10 TCID50/ml)

Time after infection (h)

| Virus | 0 h | 8 h | 24 h | 48 h |
|---|---|---|---|---|
| MTH68 | 4.8 | 4.5 | 7.0 | 7.0 |
| Mut HN(F277L)/M(G165W) | 5.0 | 4.3 | 7.8 | 8.3 |
| rgMTH68 | 5.0 | 4.0 | 7.0 | 7.5 |
| rgMut HN(L277F) | 5.0 | 4.3 | 7.0 | 7.3 |
| rgMut M(W165G) | 4.8 | 4.3 | 7.8 | 7.5 |
| rgMutHu | 5.3 | 4.5 | 8.0 | 8.0 |

### Example 4. Generation of Recombinant NDV-Strains with Enhanced Oncolytic and Immune Stimulating Properties Due to the Expression of Different Therapeutic Proteins

To this end, three different rgMut HN(F277L)/M(G165W) strains were generated, expressing the genes for:

### 1) Ipilimumab

### 2) Interleukin 12

3) Non-structural protein NS 1 of influenza A virus

### 4.1 Generation of Recombinant Viruses

Recombinant NDV-Mut HN(F277L)/M(G165W) viruses (rgNDV-Mut HN(F277L)/M(G165W)) expressing Ipilimumab, interleukin-12 or NS1 were generated by means of the previously established reverse genetics system described above. The genes encoding the heavy- and light-chain of Ipilimumab or the gene encoding interleukin 12 or the gene encoding the non-structural protein NS1 of influenza A virus were inserted into the full-length cDNA of NDV-Mut HN(F277L)/M(G165W) between the P and the M genes. To this end the open reading frames of the foreign genes were fused via a 2A sequence. The genes were provided with the necessary NDV gene-start and gene-end sequences in order to allow transcription by the vRNA polymerase.

FIG. 2 shows the final constellation of the recombinant virus that was rescued from cloned full-length cDNA by means of the NDV reverse-genetics systems.

Infectious virus was rescued for all three constructs, and virus stocks were prepared by two passages in HeLa cells. The nucleotide sequences of the inserted genes in the different recombinant viruses were verified by means of nucleotide sequence analysis and found to be correct.

Expression of Ipilimumab was determined and quantified by using a human IgG ELISA (Invitrogen). The amount of Ipilimumab that is secreted into the medium of rgMut HN(F277L)/M(G165W)-Ipilimumab infected HeLa cells was determined by analyzing the culture supernatant of 3 different infections.

As can be seen from the OD450 values given in Table 2 below, the production reached approximately 6,400-7,000 ng/ml.

TABLE 2
OD450 values of a human IgG ELISA

| Calibration samples | | | rgMutHu | rgMutHu-Ipilimumab | | | |
|---|---|---|---|---|---|---|---|
| IgG | series | series | neg | sample | sample | sample | |
| ng/ml | 1 | 2 | control | 1 | 2 | 3 | dilution |
| 100.00 | 1.131 | 1.277 | 0.155 | 1.818 | 1.770 | 1.655 | 1:2 |
| 50.00 | 0.624 | 0.737 | 0.108 | 1.754 | 1.591 | 1.471 | 1:4 |
| 25.00 | 0.351 | 0.415 | 0.082 | 1.577 | 1.503 | 1.426 | 1:8 |
| 12.50 | 0.198 | 0.232 | 0.066 | 1.425 | 1.411 | 1.321 | 1:16 |
| 6.25 | 0.134 | 0.143 | 0.121 | 1.169 | 1.067 | 1.012 | 1:32 |
| 3.12 | 0.095 | 0.099 | 0.094 | 0.874 | 0.887 | 0.908 | 1:64 |
| 0.00 | 0.081 | 0.081 | 0.080 | 0.678 | 0.601 | 0.623 | 1:128 |
| 0.00 | 0.067 | 0.081 | 0.071 | 0.469 | 0.413 | 0.408 | 1:256 |

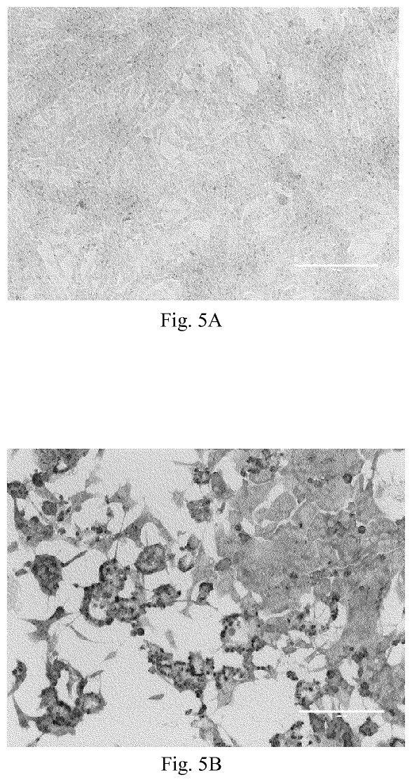

Faithfull expression of non-structural protein NS1 by rgMutHu-NS1 was verified by means of immunological staining of rgMutHu-NS1 infected monolayers using an antibody against against influenza A (H1N1) NS1 protein (FIG. 5). An expression of NS1 was only detectable in cells infected with rgMutHu-NS1 (FIG. 5B).

### REFERENCES

Blach-Olszewska et al., (1977) Why HeLa cells do not produce interferon? Arch Immunol Ther Exp (Warsz) 25:683-91.
Chng et al., (2015) Cleavage efficient 2A peptides for high level monoclonal antibody expression in CHO cells, mAbs, 7:2, 403-412; http://dx.doi.org/10.1080/19420862.2015.1008351.
Enoch et al., (1986) Activation of the Human beta-Interferon Gene Requires an Interferon-Inducible Factor, Mol. Cell. Biol. 6:801-10.
Haryadi et al., (2015) Optimization of Heavy Chain and Light Chain Signal Peptides for High Level Expression of Therapeutic Antibodies in CHO Cells. PLoS ONE 10(2): e0116878. doi:10.1371/journal.pone.0116878.
Schirrmacher, (2015) Oncolytic Newcastle disease virus as a prospective anti-cancer therapy. A biologic agent with potential to break therapy resistance. Expert Opin. Biol. Ther. 15:17 57-71
Zamarin et al., (2014) Localized oncolytic virotherapy overcomes systemic tumor resistance to immune checkpoint blockade immunotherapy. Sci. Transl. Med. 6(226).
Zamarin & Palese, (2017) Oncolytic Newcastle Disease Virus for cancer therapy: old challenges and new directions. Future Microbiol. 7: 347-67.

APPEMDIX 1
primers used for the generation of
cDNA fragments and helper-plasmids
cDNA fragments

| Fragment | Size | Primer | Sequence (5′-3′) |
|---|---|---|---|
| C1 | 3.6 kb | Noss-09 | ACGACTCACTATAGGaccaaacagagaatccgtgag |
| | | (SEQ ID No. 8) | |
| | | Noss-121 | CCGGGAAGATCCAGGgcactcttcttgcatgttac |
| | | (SEQ ID No. 9) | |
| C2 | 3.7 kb | Noss-122 | GGGCCTGCCTCACTAtggtggtaacatgcaagaag |
| | | (SEQ ID No. 10) | |
| | | Noss-123 | TGCATGTTACCACCAatgtgtcattgtatcgcttg |
| | | (SEQ ID No. 11) | |
| C3 | 5.7 kb | Noss-125 | CAAGAAGGGAGATACgtaatatacaagcgatacaatg |
| | | (SEQ ID No. 12) | |
| | | Noss-126 | TCGCTTGTATATTACttgttgtagcaaagagcacc |
| | | (SEQ ID No. 13) | |
| C8 | 2.0 kb | Noss-133 | GGCCTGGATCTTCCCattatgctgtctgtatacggtgc |
| | | (SEQ ID No. 14) | |
| | | Noss-10 | ATGCCATGCCGACCCaccaaacaaagacttggtgaatg |
| | | (SEQ ID No. 15) | |
| iPCR | 2.5 kb | Noss-17 | CCTATAGTGAGTCGTATTAATTTC |
| (StuI) | | (SEQ ID No. 16) | |
| pOLTV5 | | Noss-128 | CCTGGATCTTCCCGGGTCGG |
| | | (SEQ ID No. 17) | |
| iPCR | 2.5 kb | Noss-137 | GGGTCGGCATGGCATCTCCACC |
| pOLTV5 | | (SEQ ID No. 18) | |
| (SmaI) | | Noss-138 | GGGAAGATCCAGGCCTATAGTG |
| | | (SEQ ID No. 19) | |

What is claimed is:

1. A recombinant Newcastle Disease Virus (NDV), carrying as a foreign gene at least one gene selected from the group consisting of:
a gene encoding an antibody directed to the surface protein CTLA-4 or an antigen-binding part thereof directed to the surface protein CTLA-4 (anti-CTLA-4), such as Ipilimumab, an antigen-binding part of Ipilimumab, a functional analog of Ipilimumab or a functional analog of an antigen-binding part of Ipilimumab,
a gene encoding a protein which improves the cellular immune response and improves the ability of T cells to enter tumor cells, or a part thereof which improves the cellular immune response and improves the ability of T cells to enter tumor cells, such as interleukin-12 (IL-12), a part of interleukin-12, a functional analog of interleukin-12 or a functional analog of a part of interleukin-12,
a gene encoding a protein with the ability to modulate the virus replication cycle, or a part thereof with the ability to modulate the virus replication cycle, such as the non-structural protein NS1 of influenza A virus, a part of the non-structural protein NS1 of influenza A virus, a functional analog of the non-structural protein NS1 of influenza A virus or a functional analog of a part of the non-structural protein NS1 of influenza A virus, and
any combination of these genes, parts or funcational analogs thereof, wherein
said recombinant NDV having a mutation in the HN gene leading to a change in the amino acid sequence of the respective gene product and providing the NDV with an increased replication capability in a human cancer cell as compared to an otherwise identical NDV not having the said mutation in the HN gene, preferably allowing replication of said NDV in a human cancer cell in an at least 2-fold higher level.

2. The recombinant NDV according to claim 1, wherein the NDV comprises a mutated HN gene and the encoded hemagglutinin-neuramidase with an amino acid substitution at amino acid position 277 to an amino acid with a hydrophobic side chain other than phenylalanine, and wherein phenylalanine (F) is substituted to leucine (L) at amino acid position 277 of the HN gene.

3. The recombinant NDV according to claim 2, wherein the NDV further comprises a mutation in the M gene and the thus encoded matrix protein, wherein the mutated M gene encodes a matrix protein with an amino acid substitution at amino acid position 165 to an amino acid with an aromatic side chain, and wherein glycine (G) is substituted to tryptophane (W) at amino acid position 165 of the M gene.

4. The recombinant NDV according to claim 1, wherein the NDV for carrying the at least one foreign gene is derived from NDV strain MTH-68/H (SEQ ID No. 1).

5. The recombinant NDV according to claim 1, wherein the recombinant NDV is encoded by and/or comprises at least one of the nucleic acids according to SEQ ID No. 2 to 5 or parts thereof, or the recombinant NDV is encoded by and/or comprises a nucleic acid having a sequence identity of at least 75% to one of SEQ ID No. 2 to 5.

6. The recombinant NDV according to claim 1 for use in medicine.

7. The recombinant NDV according to claim 1 for use in a method of treating cancer in a subject considered in need thereof, in particular for the treatment of one or more indications selected from the group consisting of brain tumors, like glioblastoma, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.

8. The recombinant NDV for use according to claim 7, wherein said subject is a mammalian animal or a human subject.

9. A nucleic acid encoding a recombinant NDV, the nucleic acid comprising a transgenic construct, wherein said transgenic construct encodes:
an antibody directed to the surface protein CTLA-4 or an antigen-binding part thereof directed to the surface protein CTLA-4 (anti-CTLA-4), such as Ipilimumab, an antigen-binding part of Ipilimumab, a functional analog of Ipilimumab or a functional analog of a part of Ipilimumab,
a gene encoding a protein which improves the cellular immune response and improves the ability of T cells to enter tumor cells, or a part thereof which improves the cellular immune response and improves the ability of T cells to enter tumor cells, such as interleukin-12 (IL-12), a part of interleukin-12, a functional analog of interleukin-12 or a functional analog of a part of interleukin-12,
a gene encoding a protein with the ability to modulate the virus replication cycle, or a part thereof with the ability to modulate the virus replication cycle, such as the non-structural protein NS1 of influenza A virus, a part of the non-structural protein NS1 of influenza A virus, a functional analog of the non-structural protein NS1 of influenza A virus or a functional analog of a part of the non-structural protein NS1 of influenza A virus, and/or
any combination of these genes, parts or functional analogs thereof, wherein
the nucleic acid in addition having a mutation in the HN gene, said mutation allowing replication of the NDV in a cancer cell to a higher level than replication of an otherwise identical NDV not having the said mutation in the HN gene.

10. The nucleic acid according to claim 9, wherein the sequence encoding the recombinant NDV comprises a mutated HN gene encoding hemagglutinin-neuramidase with an amino acid substitution at position 277 to an amino acid with a hydrophobic side chain other than phenylalanine, and wherein the mutated HN gene encodes HN^{F277L}.

11. The nucleic acid according to claim 9, wherein the sequence encoding the recombinant NDV further comprises a mutation in the M gene, wherein the encoded mutated M gene encodes a matrix protein with an amino acid substitution at position 165 to an amino acid with an aromatic side chain, and wherein the mutated M gene encodes M^{G165W}.

12. The nucleic acid according to claim 9, wherein the sequence encoding the NDV for carrying the transgenic construct is derived from NDV strain MTH-68/H (SEQ ID No. 1).

13. The nucleic acid according to claim 9, wherein the nucleic acid consists of or comprises at least one of the nucleic acids according to SEQ ID No. 2 to 5 or parts thereof, or the nucleic acid consists of or comprises at least one of the nucleic acids having a sequence identity of at least 75% to one of SEQ ID No. 2 to 5.

14. The nucleic acid according to claim 9, wherein the nucleic acid is derived from a recombinant NDV according to claim 1.

15. A method for preparing a rgNDV, having improved replication in a cancer cell over a parent NDV, said method comprising the steps:
a. providing a nucleic acid construct encoding a HN gene with a mutation, wherein the mutation in the HN gene leads to a change in the expression of the hemagglutinin-neuraminidase, wherein the amino acid, particularly phenylalanine (F), in position 277 is substituted, wherein the amino acid in position 277 is substituted to an amino acid with a hydrophobic side chain, and wherein the amino acid in position 277 is substituted to leucine (L) at position 277 of the HN gene,
b. providing a nucleic acid encoding a rgNDV further comprises a transgenic construct encoding:
an antibody directed to the surface protein CTLA-4 or an antigen-binding part thereof directed to the surface protein CTLA-4 (anti-CTLA-4), such as Ipilimumab, an antigen-binding part of Ipilimumab, a functional analog of Ipilimumab or a functional analog of a part of Ipilimumab,
a protein which improves the cellular immune response and improves the ability of T cells to enter tumor cells or a part thereof which improves the cellular immune response and improves the ability of T cells to enter tumor cells, such as interleukin-12 (IL-12), a part of interleukin-12, a functional analog of interleukin-12 or a functional analog of a part of interleukin-12,
a protein with the ability to modulate the virus replication cycle or a part thereof with the ability to modulate the virus replication cycle, such as the non-structural protein NS1 of influenza A virus, a part of the non-structural protein NS1 of influenza A virus, a functional analog of the non-structural protein NS1 of influenza A virus or a functional analog of a part of the non-structural protein NS1 of influenza A, and
any combination of these proteins,
c. incorporating said nucleic acid construct with said mutation in a nucleic acid encoding a rgNDV comprising the transgenic construct to achieve a nucleic acid according to claim 9,
d. using said nucleic acid obtained in step c. encoding a recombinant and mutated rgNDV to produce infectious rgNDV,
e. comparing the replication characteristics in cancer cells of the rgNDV of step d. with the replication characteristics of said parent NDV, and
f. selecting said rgNDV for further use when it shows improved replication characteristics over said parent NDV.

16. The method according to claim 15, wherein the nucleic acid encoding the rgNDV of step b carries a mutation in the F gene, wherein said mutation is capable of improving oncolytic potential of said rgNDV, and wherein said mutation in the F gene comprises or is a mutation which encodes a fusion protein having an amino acid substitution in position 289, particularly a L289A substitution.

17. The method according to claim 15, wherein the nucleic acid encoding a rgNDV of step b further carries a mutation in the RNA-editing sequence of the P gene that abolishes and/or decreases the expression of the V protein.

18. A pharmaceutical formulation comprising particles of the recombinant NDV according to claim 1 and/or a nucleic acid according to claim 9, for use in oncological treatment of a subject considered in need thereof, in particular for the treatment of one or more indications selected from the group consisting of brain tumors, bone tumors, like osteosarcoma and/or Ewing's sarcoma, soft tissue tumors, like rhabdomyosarcoma, gynecological tumors, like breast cancer, ovary cancer and/or cervix cancer, gastrointestinal tumors, like esophageal tumors, stomach tumors, colon tumors, pancreas tumors, prostate tumors, lung tumors, ear, nose, throat tumors, tongue tumors, and skin tumors, like melanoma.


SEQUENCE LISTING (25 sequences)

SEQ ID NO: 1 - DNA, 15186 bp, Newcastle disease virus
  STRAIN MTH-68/H
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatccctgggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggtttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 2 - DNA, 15186 bp, Newcastle disease virus
  Mut HN (F277L)/M(G165W)
accaaacagagaatccgtgagctacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcatccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaggggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctctgacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtcttttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggcatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 3 - DNA, 17436 bp, Newcastle disease virus
  Mut HN (F277L)/M(G165W)-Ipilimumab
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggagtttgggctgtcatgggtctttctggtcgctctgtttaggggggtgcagtgtcaggtgcagctggtcgagtcaggggggggcgtggtgcagccaggcaggtccctgaggctgtcttgcgcagccagcggcttcacctttagctcctacacaatgcactgggtgcggcaggcacctggcaagggcctggagtgggtgaccttcatctcctatgacggcaacaataagtactatgccgattctgtgaagggccggtttaccatctccagagacaactctaagaatacactgtacctgcagatgaacagcctgagggccgaggacaccgccatctactattgcgcaaggacaggatggctgggacctttcgattattggggccagggcaccctggtgacagtgtctagcgcctctaccaagggcccaagcgtgtttcccctggccccttcctctaagtccacctctggaggcacagccgccctgggctgtctggtgaaggattacttccctgagccagtgaccgtgagctggaactccggcgccctgaccagcggagtgcacacatttccagccgtgctgcagagcagcggcctgtactccctgtctagcgtggtgaccgtgccctcctctagcctgggcacccagacatatatctgcaacgtgaatcacaagcctagcaatacaaaggtggacaagcgggtggagccaaagtcctgtgataagacccacacatgcccaccttgtccagcacctgagctgctgggaggaccatccgtgttcctgtttccacccaagcccaaggacaccctgatgatcagccggacccctgaggtgacatgcgtggtggtggacgtgagccacgaggaccccgaggtgaagttcaactggtacgtggatggcgtggaggtgcacaatgccaagaccaagccccgggaggagcagtacaactccacctatagagtggtgtctgtgctgacagtgctgcaccaggactggctgaacggcaaggagtataagtgcaaggtgtccaataaggccctgccagcccccatcgagaagaccatctctaaggcaaagggacagccacgggagccacaggtgtacacactgcctcccagccgggacgagctgaccaagaaccaggtgagcctgacatgtctggtgaagggcttctatcccagcgacatcgccgtggagtgggagtccaatggccagcctgagaacaattacaagaccacaccccctgtgctggactctgatggcagcttctttctgtattctaagctgaccgtggacaagagcagatggcagcagggcaacgtgtttagctgctccgtgatgcacgaggccctgcacaatcactacacacagaagtctctgagcctgtcccctggcaagcggagaaagaggggcagcggagagggaaggggctccctgctgacctgtggcgatgtggaggagaatcctggcccaatgaagtatctgctgccaacagctgccgccggcctgctgctgctggccgcacagcccgctatggccgagatcgtgctgacccagagcccaggcacactgtctctgagccctggagagagggccaccctgtcctgcagagcctcccagtctgtgggctcctcttacctggcctggtatcagcagaagcctggccaggccccaaggctgctgatctacggagccttcagcagggccaccggcatccccgaccgcttcagcggctccggctctggcacagacttcaccctgacaatctccaggctggagcccgaggacttcgccgtgtactattgccagcagtacggcagctccccttggacctttggccagggcacaaaggtggagatcaagaggaccgtggcagcaccatccgtgttcatctttccaccctctgacgagcagctgaagagcggcacagcctccgtggtgtgcctgctgaacaacttctacccccgcgaggccaaggtgcagtggaaggtggataacgccctgcagtccggcaattctcaggagagcgtgaccgagcaggactccaaggattctacatatagcctgtctagcaccctgacactgagcaaggccgattacgagaagcacaaggtgtatgcctgtgaggtcacccaccagggactgtcatcacccgtcaccaaaagtttcaatagaggagagtgctgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 4 - DNA, 16860 bp, Newcastle disease virus
  Mut HN (F277L)/M(G165W)-IL-12
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggggcatcagcagctggtcatctcctggtttagcctggtgttcctggcaagtcctctggtcgcaatctgggaactgaaaaaagacgtgtacgtggtggagctggactggtatcctgatgccccaggcgagatggtggtgctgacctgtgacacacccgaggaggatggcatcacctggacactggatcagagcagcgaggtgctgggcagcggcaagaccctgacaatccaggtgaaggagttcggcgacgcaggacagtacacctgccacaagggaggagaggtgctgagccactccctgctgctgctgcacaagaaggaggatggcatctggagcacagacatcctgaaggatcagaaggagccaaagaacaagaccttcctgaggtgcgaggccaagaattattccggccgctttacctgttggtggctgaccacaatcagcaccgatctgacattttccgtgaagtctagccggggctcctctgacccccagggagtgacatgtggagcagccaccctgagcgccgagcgggtgagaggcgataacaaggagtacgagtattccgtggagtgccaggaggactctgcctgtcctgcagcagaggagtccctgccaatcgaagtgatggtggatgccgtgcacaagctgaagtacgagaattatacaagctccttctttatccgggacatcatcaagcctgatccccctaagaacctgcagctgaagcccctgaagaacagccggcaggtggaggtgagctgggagtacccagacacctggtccacaccccactcctatttctctctgaccttttgcgtgcaggtgcagggcaagtctaagcgggagaagaaggacagagtgttcaccgataagacaagcgccaccgtgatctgtaggaagaacgcctctatcagcgtgagggcccaggatcgctactattctagctcctggagcgagtgggcctccgtgccctgctctgtgcctggagtgggcgtgcccggagtgggcgccagaaatctgcctgtggcaaccccagaccccggaatgttcccttgtctgcaccacagccagaacctgctgcgggccgtgtccaatatgctgcagaaggccagacagacactggagttttacccctgcacctccgaggagatcgaccacgaggacatcacaaaggataagacctctacagtggaggcctgcctgcctctggagctgaccaagaacgagagctgtctgaacagccgggaaaccagcttcatcaccaacggcagctgcctggcctcccgcaagacatcttttatgatggccctgtgcctgtctagcatctacgaggacctgaagatgtatcaggtggagttcaagaccatgaacgccaagctgctgatggacccaaagaggcagattttcctggaccagaatatgctggccgtgatcgacgagctgatgcaggccctgaacttcaatagcgagacagtgccacagaagtcctctctggaggagcccgacttttacaagaccaagatcaagctgtgcatcctgctgcacgctttccgaatccgagccgtgacaatcgacagagtgatgtcctacctgaatgcctcctaatgagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 5 - DNA, 15906 bp, Newcastle disease virus
  Mut HN (F277L)/M(G165W)-NS1
accaaacagagaatccgtgagatacgataaaaggcgaaggagcaatcgaagtcgtacgggtagaaggtgtgaatctcgagtgcgagaccgaagcttaaactcgagagagccttctgccaacatgtcttccgtatttgacgagtatgaacagctcctcgcggctcagactcgccctaatggagctcacggaggaggagagaaggggagcaccttaaaagttgaagtcccggtattcactcttaacagtgacgacccagaagatagatggaactttgcagtattctgtcttaggattgctgttagcgaagatgccaacaaaccactcagacaaggtgctctcatatctctcttatgctcccattctcaagtgatgaggaaccatgtcgcccttgcagggaaacagaatgaggccacactggctgttcttgagatcgatggtttcaccaacagcgtgcctcaatttaacaacaggagtggagtgtctgaggagagagcacagagattcttgatgatagcagggtccctccctcgggcatgcagcaacggtaccccgttcatcacagctggggttgaagatgatgcaccagaagacatcactgatactctggaaagaatcatctctatccaggctcaagtatgggtcacggtagcaaaggccatgactgcatatgagacagcggatgagtcggaaacaagaagaatcaataagtatatgcagcaaggcagagtccaaaagaagtacatcctccaccccgtatgtaggagcgcaattcaactcacgattagacaatctctggcagtccgcattttcttggttagcgagcttaagagaggccgcaacacggcaggtggaagttccacttattatagcttggtaggggacatagactcatacatcaggaacaccgggcttactgcattcttcctgacactcaggtatggaattaacaccaagacatcagcccttgcactcagcagcctcgcaggcgatatccaaaaaatgaagcagctcatgcgtctatatcggatgaaaggagataatgcgccgtacatgacactgctcggtgacagtgaccagatgagctttgcaccggctgagtatgcacaactctactcttttgcaatgggtatggcatccgtcctagacaaaggaactggcaagtaccagttcgccagagactttatgagcacatcattctggagacttggagtagagtatgctcgggctcagggaagcagcatcaatgaggatatggctgccgagctaaagctgaccccagcggcaaggaggggcctggcagctgctgcccaacgagcgtccgaggagaccggcagcatggacattcccactcaacaagctggggtcctcacagggctcagcgacggaggccctcaagccccacaaggtggactgaacaggtcgcaagggcaaccggacgccggagatggggagacccaatttttagatttgatgagagcagtggcaaatagtatgagagaagcgccaaactctgtgcagaacaccactcagcaggagcctccatctactcctgggccatctcaagacaacgacaccgactggggatactgaccgacaacactcagcctgcctccatcgaattatctcgattcttctgcctgtaacctaacccctggtccacagtctcacacggccaaacccgcaaacgagcccccccctctctccccccccctccagccacacggctccaaccgcccaaaacaacacgggcacaactcgactcactcataatctacacagagccgaagatcttagaaaaaaatacgggtagaagagagacattcagagatcaggacaaatcatcagggtctccgctctcccctctacccagcagaccagagtgaagatggccaccttcacggatgcagagatcgacgatctatttgaaaccagtggaactgtcattgacagcataattacggcccaaggcaacccagtagagactgttggaaggagcgcaatcccacaaggcaaaactaaagcattgagtgcagcatgggagaaacatggaagcacccaaccgccggccggccaagacacccctgatcgaatggacagatcagacaaacaaccatccccacccgaacaggcaaccacacacaacagcccgccagccacatccaccgaccagccccccacccaggccgcaggcgaagccggtgacacacagctcaagactggagcaagcaactccctcctgtcgatgctcgacaagcttagcaataaatcgtccaatgctaaaaagggcccacggtcgagtccccaggaagggcaccaccaacctccgacccagcagcaagggagtcaacagagccgcggaaacagtcaggagagatcgcaaaaccaggtcaaggccgcctctggagaccagggcacagacgcgaacacagcatatcatggacaatggaaggagtcacaaccatcagctggtgcaatccctcatgctctccggtcagggcagagccaagccaatactcctgcatctgtggatcatgtccagctacctgtcgactttgtgcaggcgatgatgtctatgatggaggcgatatcacagaaggtaaataaagtcgactatcagctagaccttgtctcaaagcagacatcctccatccccatgatgcgatctgaaatccaacagctgaaaacatccgttgcggtcatggaagccaacctgggcatgatgaagattctggaccctggttgtgctaacgtttcatctttaagtgacctacgggcggtcgcccgctctcacccagttttagtttcaggccccggagacccatctccttatgtgacacaagggggtgaattgatactcaataaactctcgcaaccagtgcaacatccttctgagttgattaaacctgccacgataagcgggcctgatataggagtggagaaggacactgtccgtgcattgatcacctcacgcccaatgcatccgagctcttcagctaagctcctgagtaagctggatgcagccgggtcgatagaagaaattaggaaaatcaaacgccttgcgctgaatggctgatcactactacaacccgcaacgggttcccgtttatctattgtcacaaggaacccgccccgaagcttaagaaaaaatacgggtagaagcagtttgagagctccgttcgccaccatggatagcaataccatgtcctcctttcaggttgactgtttcctgtggcatattcgcaagcgtttcgccgataatggtctgggtgacgccccgttcctggaccgtctgcgtcgcgatcagaagagcctgaaaggccgcggtaacaccctgggcctggacatcgaaaccgcgaccctggtgggcaagcagatcgtggagtggattctgaaagaggaaagcagcgaaaccctgcgtatgaccattgccagcgtgccgaccagccgttacctgagcgacatgaccctggaggaaatgagccgtgattggttcatgctgatgccgcgccagaagatcattggtccactgtgcgtgcgtctggaccaggctatcatggagaagaacattgtgctgaaagccaacttcagcgtgatctttaaccgtctggaaaccctgattctgctgcgcgcctttaccgaggaaggcgcgatcgtgggcgagattagcccgctgccgagcctgccaggtcacacctatgaagatgtgaaaaatgccgtgggcgtgctgattggcggtctggagtggaacggtaacaccgtgcgtgtgagcgaaaacattcagcgcttcgcatggcgtaactgtgatgagaatggtcgtccaagcctgccaccggaacaaaaataagctgaccgagccctcctctataaacccaagattcaaggctccaagcgataaccttctcccgcctcttccatcccactgaatgatcgcgcagctgcaattaatctagcaacattaaagattaagaaaaaatacgggtagaatcggagtgcaccgattgtgccaagatggactcatctaggacaattgggttatactttgattctgcccttccttctagcaacctgttagcattcccgattgtcttacgagatgtaggagatggaaagaagcaaattactccgcaatataggatccggcgtcttgactcgtggacagacagtaaagaagactcggtattcatcactacctacggattcatcttccaggttgggaatgaagaagtcactgtcggcatgatcaatgataatcccaagcgagagttactctccgctgcgatgctctgcctaggaagcgtcccgaatgttggagatcctgttgagctggcaagggcctgcctcactatggtggtaacatgcaagaagagtgcaactaatactgagagaatggtcttctcagtagtgcaggcaccccgggtgctgcaaagctgtagggtcgtggcagacaaatactcgtcagtgaatgcagttaagcacgtgaaagcaccagagaagatcccttggagtgaaaccctagagtacaaggtgaattttgtctctttgactgtggtgccgaagaaggatgtctacaagatcccaaccgcagtattgaaggtatccggctcgagcctgtataatcttgcgctcaatgtcactattgatgtggaggtagacccaaagagcccgttagtcaagtccctttctaggtccgacagtggatactatgctaatctcttcttacatatcggacttatgtccactgtagataagaggggaaagaaagtgacatttgaccagctggagaggaagataaggagacttgatctatctgtcgggctcagtgatgtgctcggaccttctgtgcttgtgaaggcgagaggtgcacggactaggctgctggcacctttcttctctaacagcgggacagcctgctaccctatagcaaatgcctctcctcaggtagctaagatactctggagtcagaccgcgtgcctgcggagtgtaaaaatcattattcaagcgggcacccaacgcgctgtcgcagtgactgctgaccatgaggttacctctactaagatagagaagaggcataccatcgctaaatacaatcccttcaagaaataggccgtatccctgagactgcaattcacccgccttcccaaaacaccatgacaccagataatgatctgtcttgattacttacagttagtttccctgtctatcaaattagaaaaaacacgggtagaagagttcggatcccggccggcgcaccaaaagcgcaagatgggccccagatcttctaccaggatcccagtacctctaatgctgaccatacggatcacgctggcactgagttatgtccgtctgacaagttctcttgatggcaggcctcttgcagccgcagggatcgtggtaacaggggataaagcagttaacatatacacctcatcccagacagggtcaatcatagtcaagttactcccaaatatgcccaaggacaaagaggcatgtgcaaaagccccattggaggcttacaacaggacactgactactttgcttaccccccttggtgattctatccgcaggatacaagagtctgtgactacatccggaggaaggagacagagacgctttataggtgccattattggcagtgtagctctaggggttgcaacagctgcacagataacggcagcctcggctctgatacaagccaaccagaatgctgctaacatcctccggcttaaggagagcattgctgcaaccaatgaagctgtacacgaggtcactggcggattgtcacagttagcagtggcagttggaaagatgcagcaatttgttaatgaccagtttaataacacagctcaagagttggactgtataaaaattacacagcaggttggtgtagaactcaacttatacttaactgaattgactacagtgttcgggccacaaatcacttcccctgccttaactcagttgactgttcaggctctttacaatctggctggtggtaatgtagattacttgttgactaagttaggtgtagggaacaaccagctcagctcattgattggtagcggcttgatcaccggtaaccctattttttacgactcacagactcaactcttgggcatacaggtgactttaccctcagtcgggaacctaaataatatgcgtgccacctacttggagaccttgtctgtaagcacaacaaagggatttgcctcagcacttgtcccaaaagtagtgacacaggtcggttctgtgatagaagagcttgacacctcatactgtatagaagctgatttggatttatattgtacaagaatagtgacattccctatgtcccctggtatttattcctgtttgagcggcaatacatcggcttgcatgtattcaaggactgaaggcgcacttactacaccatacatgactctcaaaggctcagttgttgccaattgccagatgacaacatgtagatgtgcagaccccccgggtatcatatcacaaaattatggagaggctgtgtctctaatagataagcactcatgcaatgtcgtatccttagacgggataactttgaggctcagtggggaatttgatgcaacttatcaaaagaatatctcaatattagattctcaagtactagtgacaggcaatctcgatatctcaactgagcttgggaatgtcaaccactcaataagtaatgctttggataagttagaggaaagcaacagcaaactagacaaagtcaatgtcaaactgaccagcacatctgctctcattacctatattgttttaactgtcatatctcttgttcttggtatgcttagcctggttctagcatgctatctgatgtacaagcaaaaagcgcaacgaaagaccttgttgtggcttggaaataataccctagatcagatgagagctactacaaaaatgtgaatgcagatgagaggcagaggtatccccaatagcaatctgtgtgtcaattctggcagcctgttaatcagaagaattaagaaaaaactaccggatgtaggtgaacaaaagggaatatacgggtagaacggcctgagaggccacccctcaatcgggagccaggccccactacgtccgctctaccgcatcaccaacagcagtcttcagtcatggacagcgcagttagccaagttgcgctagagaatgatagaagagaagcgaataatacatggcgcttggttttccggatcgcagccttacttttactggtaataaccttagccgtctctgcagtcgccctggcatatagtatggaggctagcacacctggcgaccttgtaagcataccgactgcgatctctagggcagaggaaaggattacatctgcactcggttccaatcaagatgtagtagataggatatacaagcaagtggcccttgagtctccattggcactgctaaataccgaatctataattatgaatgcaataacgtctctctcttatcaaatcaatggagccacaaataatagcgggtgtggggcacctgttcatgacccagattacatcggggggataggtcaagaacttattgtagatgatacgagtgacgtcacatcattttatccctctgcattccaagaacacctgaattttatcccggcgcccactacaggatcaggctgcactcggataccctcattcgacatgagtgctacccattactgttacactcacaatgtgatattgtctggctgcagagatcactcacactcacatcagtatttagcacttggtgtgcttcggacatctgcaacagggagggtattcttttctactctgcgttccatcaacttggatgacgcccaaaatcggaagtcttgcagtgtgagtgcaactcctttaggttgtgatatgctgtgctctaaaatcacagagactgaggaagaggattataaatcagttatccccacatcgatggtacatggaaggttagggcttgacggccaataccatgagaaggacctagacgtcacaacactatttagggactgggtggcaaattacccaggagtaggaggtgggtcttttattaacaatcgcgtatggttcccggtctacggagggctaaaacccagctcgcctagtgacactgcacaagaagggagatacgtaatatacaagcgatacaatgacacatgcccggatgagcaagattaccagattcggatggctaagtcatcgtataagcctaggcggtttggtggaaaacgcgtacagcaggccatcctatccatcaaggtgtcaacatccttgggtgaggacccggtgttgactgtaccgcctaatacggtcgcactcatgggggccgaaggcagagttctcacagtggggacatctcatttcttatatcagcgagggtcatcatacttctctcccgctttgttataccctatgacagtcaacaacaaaacagccactcttcataatccttatacattcaatgctttcactcggccaggtagtgtcccttgccaggcttcagcaagatgccccaactcatgtgttaccggagtctatactgatccatatcccttagtcttccataggaaccacaccttgcgaggggtattcggaacaatgcttgatgacgaacaagcaagactcaaccctgtatctgcagtatttgataacatatcccgcagtcgcataactcgggtaagttcaagcagtaccagggcagcatacacgacatcaacgtgttttaaagttgtcaagaccaataaaacctattgcctcagcattgcagaaatatccaataccctcttcggggaattcagaattgtccccttactagttgagattctcaaggatggtggggtttaagaagctaggaatggtcggttgagtcaaccgtgagaaggcggggaagatgatattgcatcatctatcttctgtaacaccaagaatcaaatcgaataccagtgcgagctcgaatcctacgctgccagtcagccataatcggctggtgctaatgtggttagcctgaatcttgtcgatagtcacttgattaagaaaaaatgtggacggtagtgaaatataaggcaaagcagctcacagaggacagcacgggtaggacatggcgggctccggacccgaaagggcagagcatcagattatcctaccagagtcacatctgtcctcaccattggtcaagcataaattgctttattactggaaattaactgggctaccgcttcctgacgaatgtgacttcgaccatcttattatcagccgacaatggaagaaggtacttgaatcggccacccctgacattgagagaatgataaaactagggcgggcagtacaccagactctcaaccacaattccaggataaccggagtactccatccccggtgtttagaggaactggctagtattgaggttcctgattcaaccaacaaatttcggaagatcgaaaaaaagatccagattcacaacacaaggtatggagaactgttcacaagattatgcacgcatgtagaaaagaaactattggggtcatcttggtctaacaatgtcccacggtcagaggaattcaacagcatccgtacagatccggcattttggtttcactcaaaatggtccacagccaaatttgcatggctccatataaaacaggtccagaggcatctgattgtagcagcaagaacaaggtccgcagtcaacaaattagtgacgctgacccataaggtaggccaaatctttgttactcctgagcttgtcattgtgacacatacagatgagaacaagttcacgtgtcttacccaggaacttgtgttgatgtatgcagatatgatggagggcagagatatggtcagcataatatcatccacggcggcacatcttaggagcttatcagagaaaattgatgatattctgcggttagtagatgctctggcaagagatttgggcaatcaagtctacgatgttgtagcactaatggagggattcgcatacggcgctgttcagctgcttgaaccgtcaggtacatttgcgggggatttcttcgcattcaacctgcaggagctcaaagatactctaaccggactcctccccaaggatatcgcagaatctgtgactcacgcaatcgcaaccatattctctggcttagaacaaaatcaagcagctgagatgttgtgcctgttgcgtctgtggggtcacccactactcgagtcccgtattgcagcaaaagcagttaggagtcaaatgtgcgcaccaaaaatggtagactttgatatgatcctccaggtattatctttctttaaaggaacaatcatcaatggatatagaaagaagaatgcaggcgtgtggccacgtgtcaagatagatacgatatacgggaaggtcatagggcagctacacgcagattctgcagagatttcacatgatgtcatgttgagggaatacaagagtttatctgcacttgaattcgagccatgtatagagtatgaccctgtcaccaatctgagcatgtttttaaaagacaaggcaatcgcacacccgaaagacaactggctcgcttcgtttaggcgaaaccttctctctgaggaccagaagaaacatgtaagagaggcaacctcaactaaccgcctcttgatagagttcttagagtcaaatgattttgatccatataaagagatggaatatctgacgacccttgagtacctaagagatgataatgtggcagtatcatactcactcaaagaaaaagaggtgaaagttaatgggcggattttcgcaaagctaacaaatagattaaggaattgtcaggtaatggcagaagggatcctagctgaccagattgcacctttcttccagggaaatggggtcattcaggatagcatatctttgactaaaagtatgctagcgatgagtcaactgtctttcaacagcaacaagaaacgtattactgactgcaaagaaagagtatcctcaaaccgcaatcatgatccgaagagcaagaatcgtcggagagttgccacttttgtaactaccgacctgcaaaagtattgtctcaattggagatatcagacagtcaagctgttcgctcatgccatcaatcagctgatgggcttacctcacttcttcgagtggattcatcttagactaatggatactacgatgtttgtaggggaccctttcaatcctccaagtgaccctacggactgtgatctatcaagagtcccaaatgatgacatatatattgtcagtgctaggggaggcattgagggattatgccagaagctatggacaatgatctcaattgctgcaatccagcttgctgcagcaagatcacattgtcgcgttgcctgtatggtacaaggcgacaatcaagtaatagctgtaacgagagaggtaagatcagatgactccccagagatggtgttaacacaattgcatcaagccagtgataatttcttcaaggaattgattcatgtcaatcatttgatcggccataatttgaaggatcgtgaaaccatcaggtcagacacgttcttcatatacagcaaacgaatattcaaagatggagcaatactcagtcaggtcctcaaaaactcatctaaattagtgctaatatcaggcgaccttagtgaaaacactgtaatgtcttgtgccaacattgcatctactgtagcacggttatgcgagaacgggcttcctaaggatttctgttattacttaaactacttaatgagttgcgtgcagacatacttcgattccgaattttccatcaccaacaactcgcaactcgattctaaccagtcgtggatagaggacatttcttttgtgcactcatatgtcctgacccctgctcaactggggggactgagtaaccttcaatactcaaggctctacacaaggaacatcggcgacccgggaaccactgctttcgcagagatcaagagattagaggcagtggggttactgagtcctagcattatgactaacatcttaactaggccgcctggaaatggagattgggccagtctgtgcaacgatccatactcctttaattttgagactgtcgcaagcccaaatattgtccttaagaaacatacacaaagagtcctatttgaaacttgctcgaatcccttattatctggagtgcacacagaggataatgaggcagaagagaaggcattggctgaattcttgctcaatcaagaaatgattcatccacgtgtcgcgcatgctatcatggaagcaagctctgtaggtaggagaaagcaaattcaggggcttgttgacacaacaaacaccgtgattaagattgcactgactaggaggccactcggcatcaagaggctgatgcggatagtcaattactcgagcatgcatgcaatgctatttagagatgatgttttctcgtctaatagatccaaccatcccttagtctcttctgatatgtgttctcagacactggcagactatgcacggaacagaagctggtcacctttgacagggggtagaaaaatactgggtgtatctaatcctgataccatagaacttgtagagggtgagatccttagtgtcagtggagggtgcactagatgtgacagtggggatgaacagtttacttggttccatcttccaagcaatatagagctaaacgatgacaccagcaagaatcctccaatgagagtgccatatctcgggtcaaagactcaagagaggagagccgcctcgcttgcgaaaatagctcatatgtcaccacacgtgaaggcggctctaagggcgtcatctgtgttaatctgggcttatggggacaacgaaataaactggactgctgcccttaagattgcaaggtctcggtgcaacataagctcagagtatcttcgactattgtcacccttgcctacagctgggaatctccaacatagattggacgacggcataactcagatgacattcacccctgcatctctatacagggtgtcaccttacattcatatatctaatgattctcaaaggctatttactgaagaaggagtcaaagaggggaatgtggtttatcagcaaatcatgctcttgggtttgtccttaattgagtcactcttcccaatgacaacaaccaagacatatgatgaaatcacattgcacctccacagtaaatttagctgctgtatcagggaagcacctgttgcagttcctttcgagctactcggggtggcaccggaactaagggcagtaacctcaaataagtttatgtatgatcctagccctgtatcagagggagactttgcgagacttgacttagctatctttaagagttatgaacttaatttagagtcatattccacaatagagctaatgaacgttctttcaatatctagtgggaagttgattggccagtctgtggtttcttatgatgaagatacctccataaagaatgacgctataatagtgtatgacaacacacggaattggatcagcgaagctcagaattcagatgtggtccgcctattcgagtatgcggcactcgaagtgctcctcgactgttcttatcaactctactatctgagagtaagaggcctaaacaatatcgtcctgtacatgagtgatttatacaagaatatgccaggaattctactctctaatattgcagccacaatatctcaccctgtcatccattcaaggttgaatgcagtaggtctggtcaaccatgacgggtcacaccaacttgcagacacagatttcattgaaatgtctgcaaagctgctagtctcttgcactcgacgcgtggtctcaggtttacatgcagggaataagtatgacctgctgtttccatctgtcttggatgataacctaagtgagaagatgcttcagttgatatcccaattatgctgtctgtatacggtgctctttgctacaacaagagaaatcccgaaaataagaggcttatctgcagaagagaaatgttcagtacttactgagtacctactgtcagatgctgtgaaaccgttacttgggtccgagcaagtgagctctatcatgtctcccaacatagttacgttcccagccaatctgtattacatgtctaggaagagccttaatttgatcagggagagagaggacagggatactatcttggcattgttgttccctcaagaaccgctgctcgagtttcctctggtacgagatattggtgctcgtgtaaaagatccatttacccgacaacctgcggcgtttttacaagagttagatttgagtgctccggcaaggtatgacgcattcacaatcagtcaggcgcattctgaacacatattgccaaacccagaggaagatcacttagtacgatacttgttcagaggaatagggactgcgtcctcctcttggtataaggcatcccatcttctttctgtacccgaggtcagatgtgcaaggcatgggaactccttatatttagcagaaggaagtggagccatcatgagtcttctcgaattgcatataccacacgaaactatctattacaatacacttttctcgaacgagatgaaccccccacagcgacatttcggaccaactccaacacagtttctgaattcggttgtttttaggaatttacaggcggaagtaccatgcaaggatggatttgtccaggagttccgtccgttatggagagagaatacagaagaaagcgatctgacctcagataaagcagtgggatatatcacatctgtagtgccgtacaggtctgtatcattgctgcattgtgacattgaaatccctccaggatctaatcaaagcttactagatcagctggctaccaatctgtctctgattgccatgcattctgtgaaggagggcggggtcgtgattatcaaagtactgtatgcaatgggatattacttccatctactcatgaacttgtttactccatgttccacgaaaggatatattctctctaatggctatgcctgtagaggggatatggagtgttacctgatatttgtcatgggctacctaggcgggcctacatttgtacatgaggtggtgaggatggcaaaaacgctagtacagcggcatggcacacttttgtctaagtcagatgagattacactgactaggttatttacttcacagcaacagcgtgtaacagacatcctatccagccctttaccgagactaatgaagtacttgagagagaatattgatactgcactgattgaagccgggggacagcctgtccgtccattctgtgcagagagtttagtgagcacactaacagacatgactcagacaacccagatcatcgccagccacattgatacagtcattcgatctgtgatctatatggaagcagagggtgatcttgctgacacagtattcttatttaccccttacaacctctctatggacgggaaaaagagaacatcacttaaacagtgcacaagacagatcttagaggtcacaatactgggtctcagagtcaaaaatctcaataaagtaggtgatgtaatcagcctagtactcagaggtatgatttctctggaggaccttctcccactgagaacctacttgaagtgtagtacctgccctaagtatttgaaggctgtcctaggtattaccaaactcaaagaaatgttcacagacgcctctttattatacttgactcgtgctcaacaaaaattctacatgaaaactataggcaatgcagtcaagggatactatagtaactgtgactcttaaaggcaaccacatatcaataggccctctttctagccgatcgtattcttgttgacttcattataccatattagaaaaaaattgaattccgaccctttaagactcgtattcggattcaaataattatctcagaaaaaaagtgcacgtagttgttcttgattatagtcccgtcattcaccaagtctttgtttggt

SEQ ID NO: 6 - protein, 571 aa, Newcastle disease virus
  HN protein
MDSAVSQVALENDRREANNTWRLVFRIAALLLLVITLAVSAVALAYSMEASTPGDLVSIPTAISRAEERITSALGSNQDVVDRIYKQVALESPLALLNTESIIMNAITSLSYQINGATNNSGCGAPVHDPDYIGGIGQELIVDDTSDVTSFYPSAFQEHLNFIPAPTTGSGCTRIPSFDMSATHYCYTHNVILSGCRDHSHSHQYLALGVLRTSATGRVFFSTLRSINLDDAQNRKSCSVSATPLGCDMLCSKITETEEEDYKSVIPTSMVHGRLGLDGQYHEKDLDVTTLFRDWVANYPGVGGGSFINNRVWFPVYGGLKPSSPSDTAQEGRYVIYKRYNDTCPDEQDYQIRMAKSSYKPRRFGGKRVQQAILSIKVSTSLGEDPVLTVPPNTVALMGAEGRVLTVGTSHFLYQRGSSYFSPALLYPMTVNNKTATLHNPYTFNAFTRPGSVPCQASARCPNSCVTGVYTDPYPLVFHRNHTLRGVFGTMLDDEQARLNPVSAVFDNISRSRITRVSSSSTRAAYTTSTCFKVVKTNKTYCLSIAEISNTLFGEFRIVPLLVEILKDGGV

SEQ ID NO: 7 - protein, 364 aa, Newcastle disease virus
  M protein
MDSSRTIGLYFDSALPSSNLLAFPIVLRDVGDGKKQITPQYRIRRLDSWTDSKEDSVFITTYGFIFQVGNEEVTVGMINDNPKRELLSAAMLCLGSVPNVGDPVELARACLTMVVTCKKSATNTERMVFSVVQAPRVLQSCRVVADKYSSVNAVKHVKAPEKIPWSETLEYKVNFVSLTVVPKKDVYKIPTAVLKVSGSSLYNLALNVTIDVEVDPKSPLVKSLSRSDSGYYANLFLHIGLMSTVDKRGKKVTFDQLERKIRRLDLSVGLSDVLGPSVLVKARGARTRLLAPFFSNSGTACYPIANASPQVAKILWSQTACLRSVKIIIQAGTQRAVAVTADHEVTSTKIEKRHTIAKYNPFKK

SEQ ID NO: 8 - DNA, 36 bp, Artificial Sequence
  Primer
acgactcactataggaccaaacagagaatccgtgag

SEQ ID NO: 9 - DNA, 35 bp, Artificial Sequence
  Primer
ccgggaagatccagggcactcttcttgcatgttac

SEQ ID NO: 10 - DNA, 35 bp, Artificial Sequence
  Primer
gggcctgcctcactatggtggtaacatgcaagaag

SEQ ID NO: 11 - DNA, 35 bp, Artificial Sequence
  Primer
tgcatgttaccaccaatgtgtcattgtatcgcttg

SEQ ID NO: 12 - DNA, 37 bp, Artificial Sequence
  Primer
caagaagggagatacgtaatatacaagcgatacaatg

SEQ ID NO: 13 - DNA, 35 bp, Artificial Sequence
  Primer
tcgcttgtatattacttgttgtagcaaagagcacc

SEQ ID NO: 14 - DNA, 38 bp, Artificial Sequence
  Primer
ggcctggatcttcccattatgctgtctgtatacggtgc

SEQ ID NO: 15 - DNA, 38 bp, Artificial Sequence
  Primer
atgccatgccgacccaccaaacaaagacttggtgaatg

SEQ ID NO: 16 - DNA, 24 bp, Artificial Sequence
  Primer
cctatagtgagtcgtattaatttc

SEQ ID NO: 17 - DNA, 20 bp, Artificial Sequence
  Primer
cctggatcttcccgggtcgg

SEQ ID NO: 18 - DNA, 22 bp, Artificial Sequence
  Primer
gggtcggcatggcatctccacc

SEQ ID NO: 19 - DNA, 22 bp, Artificial Sequence
  Primer
gggaagatccaggcctatagtg

SEQ ID NO: 20 - DNA, 35 bp, Artificial Sequence
  Primer
ctctagagtcgacccttctgccaacatgtcttccg

SEQ ID NO: 21 - DNA, 35 bp, Artificial Sequence
  Primer
gggaagcggccgcccgtcggtcagtatccccagtc

SEQ ID NO: 22 - DNA, 37 bp, Artificial Sequence
  Primer
ctctagagtcgaccccagagtgaagatggccaccttc

SEQ ID NO: 23 - DNA, 35 bp, Artificial Sequence
  Primer
gggaagcggccgcccgtagtagtgatcagccattc

SEQ ID NO: 24 - DNA, 35 bp, Artificial Sequence
  Primer
ctctagagtcgacccgggtaggacatggcgggctc

SEQ ID NO: 25 - DNA, 37 bp, Artificial Sequence
  Primer
gggaagcggccgccctgcctttaagagtcacagttac